(19) United States
(12) Patent Application Publication
(10) Pub. No.: US 2021/0267492 A1
(43) Pub. Date: Sep. 2, 2021

(54) SYSTEMS AND METHODS FOR DETECTING A MOTOR DEVELOPMENTAL DELAY OR NEURODEVELOPMENTAL DISORDER IN AN INFANT

(71) Applicants: Katelyn Elizabeth FRY, Atlanta, GA (US); Faraz Muhammad YOUSUF, Indianapolis, IN (US); Yu-Ping CHEN, Alpharetta, GA (US); Georgia Tech Research Corporation, Atlanta, GA (US)

(72) Inventors: Katelyn Elizabeth FRY, Atlanta, GA (US); Faraz Muhammad YOUSUF, Indianapolis, IN (US); Yu-Ping CHEN, Alpharetta, GA (US); Ayanna Howard, Atlanta, GA (US)

(21) Appl. No.: 17/261,230

(22) PCT Filed: Jul. 19, 2019
(86) PCT No.: PCT/US2019/042559

§ 371 Date: Jan. 19, 2021

Related U.S. Application Data
(60) Provisional application No. 62/700,781, filed on Jul. 19, 2018.

(51) Int. Cl. A61B 5/11; A61B 5/00; G16H 50/20

(52) U.S. Cl. CPC A61B 5/1124; A61B 5/1123; A61B 5/4082; A61B 5/7267; A61B 5/1118; A61B 5/0024; A61B 5/7282; G16H 50/20; A61B 5/1121; A61B 2503/045; A61B 5/6829

(57) ABSTRACT

Systems and methods for detecting a motor developmental delay and/or neurodevelopmental disorder of an infant are described herein. An example method can include receiving motion data associated with the infant's gross motor activity; analyzing, using a machine learning algorithm, the motion data to detect a kinematic feature; comparing the kinematic feature to an expected relationship between the kinematic feature and infant age; and detecting the neurodevelopmental disorder based on the comparison. An infant sensor suit is also described herein. An example infant sensor suit can include an article of clothing; a plurality of sensors; a power source operably coupled to the sensors; and a wireless transmitter operably coupled to the sensors. The sensors, power source, and wireless transmitter can be incorporated into the article of clothing.

### CROSS-REFERENCE TO RELATED APPLICATIONS

This application claims the benefit of U.S. provisional patent application No. 62/700,781, filed on Jul. 19, 2018, and entitled “Detection of Infant Motor Activity During Spontaneous Kicking Movements for Term and Preterm Infants Using Inertial Sensors,” the disclosure of which is expressly incorporated herein by reference in its entirety.

### STATEMENT REGARDING FEDERALLY FUNDED RESEARCH

This invention was made with government support under Grant no. 1545287 awarded by the National Science Foundation. The government has certain rights in the invention.

### BACKGROUND

According to the Center for Disease Control and Prevention (CDC), one out of every ten births in the United States is considered premature (infants with a gestation period of 37 weeks or less). Despite the improved neonatal care, which has led to an increase in the number of surviving preterm infants, preterm infants are at an increased risk of developing neurodevelopmental disorders. The most common of these disorders among children is cerebral palsy (CP), which affects approximately 2 to 2.5 per 1000 live births [A. Herskind et al. “Early identification and intervention in cerebral palsy,” Developmental Medicine & Child Neurology, v. 57 no.1, pp. 29-36, 2015].

CP is a spectrum disorder that encompasses various categories of motor function disorders and varies in severity across individuals. Research has shown that interventions may improve the overall quality of life of affected individuals if CP can be reliably detected early in life [E. Rogers et al., “Smart and Connected Actuated Mobile and Sensing Suit to Encourage Motion in Developmentally Delayed Infants,” in ASME Design of Medical Devices Conf., Minneapolis, Minn., 2015; K. Subramanyam et al., “Soft Wearable Orthotic Device for Assisting Kicking Motion in Developmentally Delayed Infants,” J. of Medical Devices, v. 9 no. 3, 2015]. However, due to the variability between individual cases of CP, it is difficult to design a diagnostic test to encompass all patients [D. Bryant et al., “An infant smart-mobile system to encourage kicking movements in infants at-risk of cerebral palsy,” in IEEE Workshop on Advanced Robotics and its Social Impacts (ARSO), Austin, Tex., 2017]. Currently, detecting the development of CP in infancy requires clinical observation and documentation of functional motor milestones in combination with neurological assessments [M. Hadders-Algra, “Early diagnosis and early intervention in cerebral palsy,” Frontiers in Neurology, v. 5, 2014]. These approaches are not appropriate for the detection of CP early in life as such milestones are not typically exhibited in the first few months. Additionally, the observation of infant motor ability is typically confined to a clinical setting which limits the amount of time an infant can be observed [B. A. Smith et al., “Daily Quantity of Infant Leg Movement: Wearable Sensor Algorithm and Relationship to Walking Onset,” Sensors, v. 15 no. 8, pp. 19006-19020, 2015]. Moreover, these approaches are subjective by nature due to their dependence on infant cooperation during the observation time and opinion of the clinician. To date, an objective method for the extended observation of infant motor development and the early detection of CP is not available.

### SUMMARY

An example computer-implemented method for detecting a neurodevelopmental disorder of an infant is described herein. The method can include receiving motion data associated with the infant's gross motor activity; analyzing, using a machine learning algorithm, the motion data to detect a kinematic feature; comparing the kinematic feature to an expected relationship between the kinematic feature and infant age; and detecting the neurodevelopmental disorder based on the comparison.

Additionally, the infant's gross motor activity can include a plurality of spontaneous kicking movements.

In some implementations, the kinematic feature can be a percentage of time the infant spent in a motion state. Optionally, the motion state can be no motion, unilateral motion, or bilateral motion.

In other implementations, the kinematic feature can be kick frequency, spatiotemporal organization, inter-joint coordination, inter-limb coordination, phase lag, constrained movement duration, duration of movement, average acceleration, peak acceleration, joint angles, joint angle excursion, peak joint velocities, or intra-limb coordination.

Alternatively or additionally, the step of detecting the neurodevelopmental disorder based on the comparison can include detecting that the infant is motor developmentally delayed for the infant's age.

Alternatively or additionally, the machine learning algorithm can be a supervised or unsupervised learning algorithm. For example, the machine learning algorithm can be a thresholding algorithm, a K-nearest neighbors (KNN) algorithm, or a Gaussian mixture model (GMM).

Alternatively or additionally, the motion data can be received from one or more sensors placed at the infant's lower limb.

Alternatively or additionally, the neurodevelopmental disorder can be cerebral palsy.

An example computer-implemented method for detecting a motor developmental delay of an infant is described herein. The method can include receiving motion data associated with the infant's gross motor activity; analyzing, using a machine learning algorithm, the motion data to detect a kinematic feature; comparing the kinematic feature to an expected relationship between the kinematic feature and infant age; and detecting the motor developmental delay based on the comparison.

An example system for detecting a neurodevelopmental disorder of an infant can include a sensor configured for placement at the infant's lower limb; and a computing device operably coupled to the sensor. The computing device can include a processor and a memory operably coupled to the processor. The computing device can be configured to receive, from the sensor, motion data associated with the infant's gross motor activity; analyze, using a machine learning algorithm, the motion data to detect a kinematic feature; compare the kinematic feature to an expected relationship between the kinematic feature and infant age; and detect the neurodevelopmental disorder based on the comparison.

In some implementations, the system includes a plurality of sensors configured for placement at the infant's lower limb.

Additionally, the sensor can be configured for placement at the infant's thigh, shin, or foot. Optionally, the sensor can be configured for placement at the infant's foot.

Alternatively or additionally, the sensor is an inertial measurement unit (IMU).

An example system for detecting a motor developmental delay of an infant can include a sensor configured for placement at the infant's lower limb; and a computing device operably coupled to the sensor. The computing device can include a processor and a memory operably coupled to the processor. The computing device can be configured to receive, from the sensor, motion data associated with the infant's gross motor activity; analyze, using a machine learning algorithm, the motion data to detect a kinematic feature; compare the kinematic feature to an expected relationship between the kinematic feature and infant age; and detect the motor developmental delay based on the comparison.

An example infant sensor suit is described herein. The infant sensor suit can include an article of clothing; a plurality of sensors; a power source operably coupled to the sensors; and a wireless transmitter operably coupled to the sensors. The sensors, power source, and wireless transmitter can be incorporated into the article of clothing.

Additionally, the infant sensor suit can further include a wearable circuit that is incorporated into the article of clothing. The wearable circuit can be configured to operably couple the sensors and the power source. Optionally, the wearable circuit can include a conductive fabric or thread.

Alternatively or additionally, a respective sensor can be arranged at the infant's thigh, shin, and foot.

Alternatively or additionally, the infant sensor suit can further include a sensor pocket that is attached to the article of clothing. The sensor pocket can be configured to receive the sensors. Optionally, respective positions of the sensors are configured to be adjustable within the sensor pocket.

Alternatively or additionally, the power source can optionally be a single power source that is operably coupled to the sensors.

It should be understood that the above-described subject matter may also be implemented as a computer-controlled apparatus, a computer process, a computing system, or an article of manufacture, such as a computer-readable storage medium.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The components in the drawings are not necessarily to scale relative to each other. Like reference numerals designate corresponding parts throughout the several views.

FIG. 1 is a block diagram illustrating an example system for detecting a neurodevelopmental disorder according to an implementation described herein.

FIG. 2 is a flow diagram illustrating an example operations for detecting a neurodevelopmental disorder according to an implementation described herein.

FIG. 3 is a block diagram illustrating an example computing device.

FIG. 4A illustrates an example infant sensor suit according to an implementation described herein. FIG. 4B illustrates a soft circuit of the infant sensor suit of FIG. 4A. FIG. 4C illustrates the soft circuit of the infant sensor suit of FIG. 4A. FIG. 4D illustrates the soft circuit and a sensor pocket of the infant sensor suit of FIG. 4A. FIG. 4E illustrates a sensor container for use with the infant sensor suit of FIG. 4A. FIG. 4F illustrates a regulator container for use with the infant sensor suit of FIG. 4A.

FIG. 5 illustrates sensor placement according to the examples described herein.

FIG. 6A illustrates 50 second sample of 3-axis accelerometry data from the left foot of infant D. Time elapsed indicates time elapsed since beginning of segment. FIG. 6B illustrates codifier identified left leg motion. Portions shaded in gray indicate periods of activity. FIG. 6C illustrates activity detector with OR decision fusion method identified periods of activity. FIG. 6D illustrates activity detector with MODE decision fusion method identified periods of activity.

FIGS. 7A-7D illustrate example of individual codifier decisions (bottom three subplots, i.e., FIGS. 7B-7D) and the overall combined truth vector (FIG. 7A). At instances of disagreement between the codifiers, the most popular identifier at a particular time was chosen to construct the combined truth vector. For example, at time elapsed t=25 and t=32, there was disagreement between codifier 1 (FIG. 7B) and codifiers 2 and 3 (FIGS. 7C and 7D). As such, for the combined truth vector, the decision of codifier 2 and 3 were chosen over the decision of codifier 1.

FIG. 8 is a table with classifier performance when different combinations of sensors are present.

FIG. 9A-9C are plots for the percent of time an infant spent in the various motion states versus adjusted age rounded to the nearest half month. No motion state is shown in FIG. 9A, unilateral motion state is shown in FIG. 9B, and bilateral motion state is shown in FIG. 9C. Here, the percentages for the premature infants (indicated with the blue+markers) were plotted against the infant's adjusted age. The adjusted age is calculated by subtracting the number of weeks the infant was born before their due date from the infant's birth age.

FIG. 10A-10C are plots for the percent of time an infant spent in the various motion states versus birth age rounded to the nearest half month. No motion state is shown in FIG. 10A, unilateral motion state is shown in FIG. 10B, and bilateral motion state is shown in FIG. 10C. Here, the percentages for the premature infants (indicated with the blue+markers) were plotted against the infant's birth age. The motion states were fit with a linear model to determine which age premature infants should be considered when examining their movement breakdown.

FIG. 11A-11C are plots for the percent of time infant D spent in the various motion states versus birth age. No motion state is shown in FIG. 11A, unilateral motion state is shown in FIG. 11B, and bilateral motion state is shown in FIG. 11C. Infant D was born to term. For infant D, the general trends discussed for the entire group are observed here over multiple months. That is, the amount of time infant D spent at rest increases with age as the amount of time spent performing bilateral and unilateral motion decreased with age.

FIG. 12A-12C are plots for the percent of time infant F spent in the various motion states versus adjusted age. No motion state is shown in FIG. 12A, unilateral motion state is shown in FIG. 12B, and bilateral motion state is shown in FIG. 12C. Infant R was born 6 weeks preterm. Infant F also follows the general trends discussed for the entire group. However, there is a distinct increase in % time for unilateral motion and a distinct decrease in % time for no motion from 5.5 months to 6.5 months adjusted age that goes against the trend for the overall group.

### DETAILED DESCRIPTION

Unless defined otherwise, all technical and scientific terms used herein have the same meaning as commonly understood by one of ordinary skill in the art. Methods and materials similar or equivalent to those described herein can be used in the practice or testing of the present disclosure. As used in the specification, and in the appended claims, the singular forms “a,” “an,” “the” include plural referents unless the context clearly dictates otherwise. The term “comprising” and variations thereof as used herein is used synonymously with the term “including” and variations thereof and are open, non-limiting terms. The terms “optional” or “optionally” used herein mean that the subsequently described feature, event or circumstance may or may not occur, and that the description includes instances where said feature, event or circumstance occurs and instances where it does not. Ranges may be expressed herein as from “about” one particular value, and/or to “about” another particular value. When such a range is expressed, an aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Conventionally, the standard procedure to identify infants who have delayed motor development is to observe their repertoire of functional motor skills in a clinical setting. These observations typically produce a subjective measure of an infant's motor development. Furthermore, these observations can be limited by factors such as available clinical observation time, infant cooperation during the observation, and ease of access to a clinical facility.

The systems and methods described herein allow for the collection of infant kicking data outside of a clinical setting. The systems and methods described herein are more widely accessible and provide longer periods of observation compared to typical clinical observation times. Finally, data collected with the systems and methods described herein provide a more quantitative measure of infant motor development.

### Example Systems

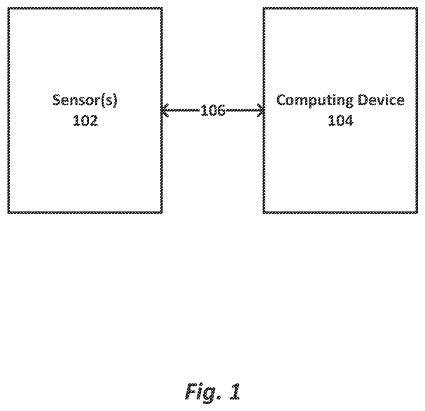

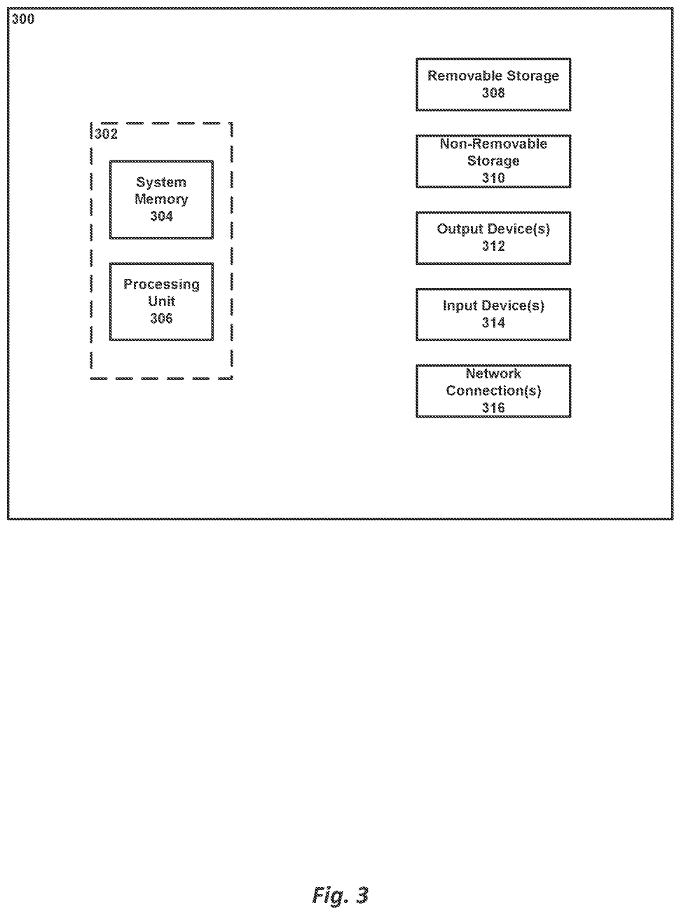

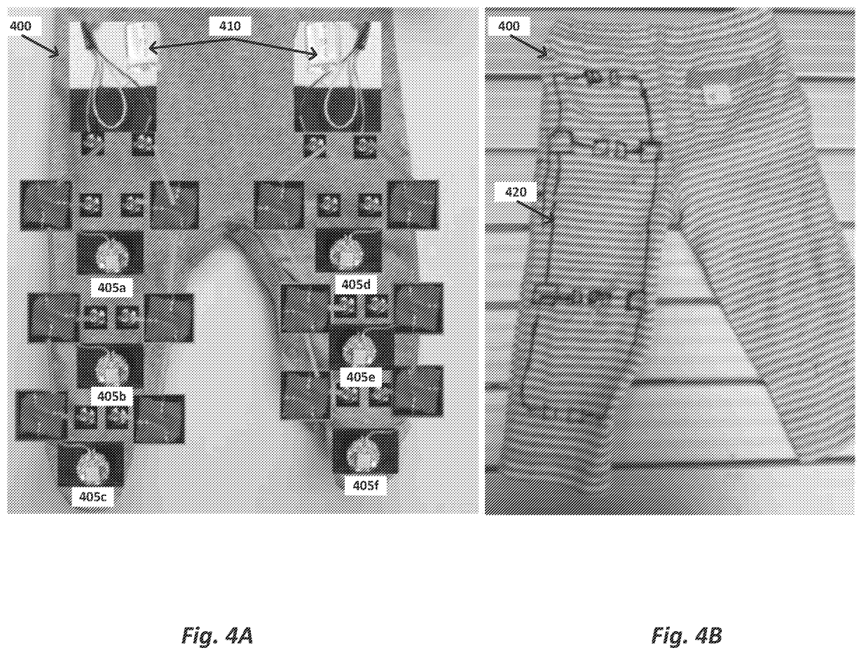

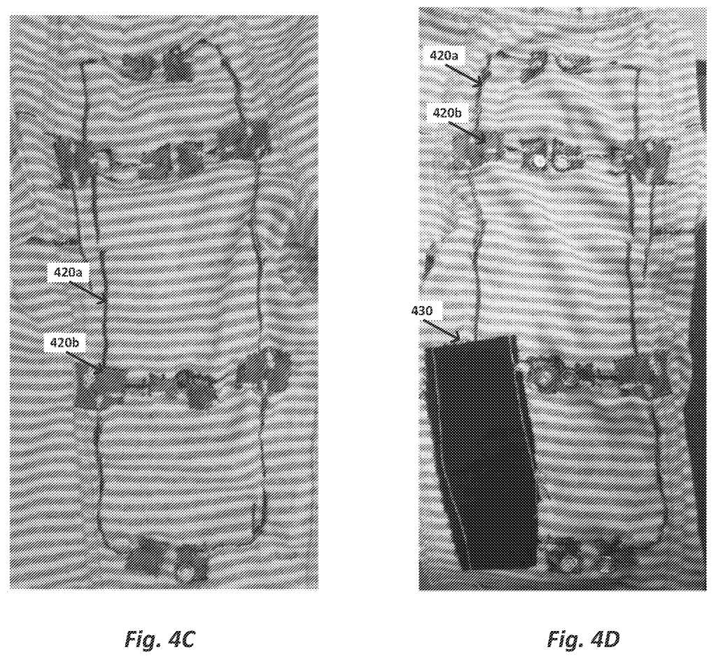

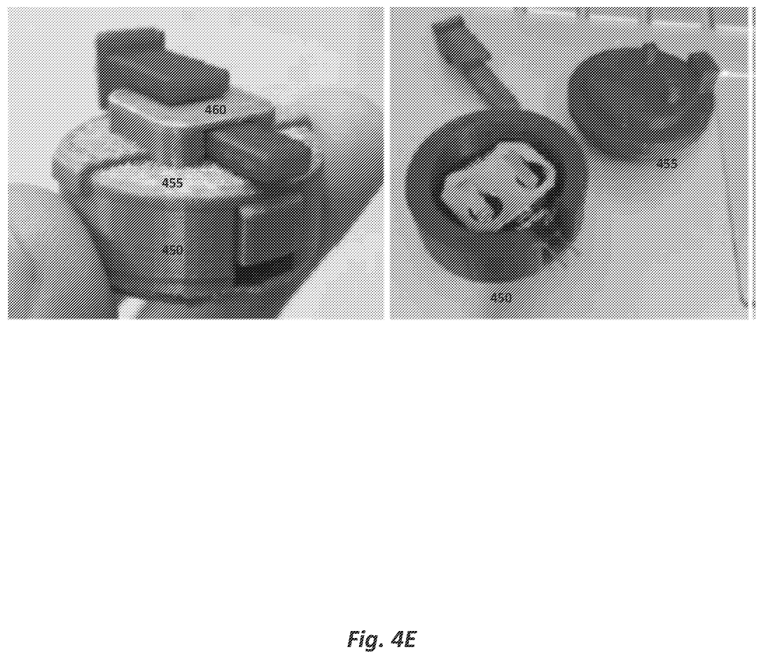

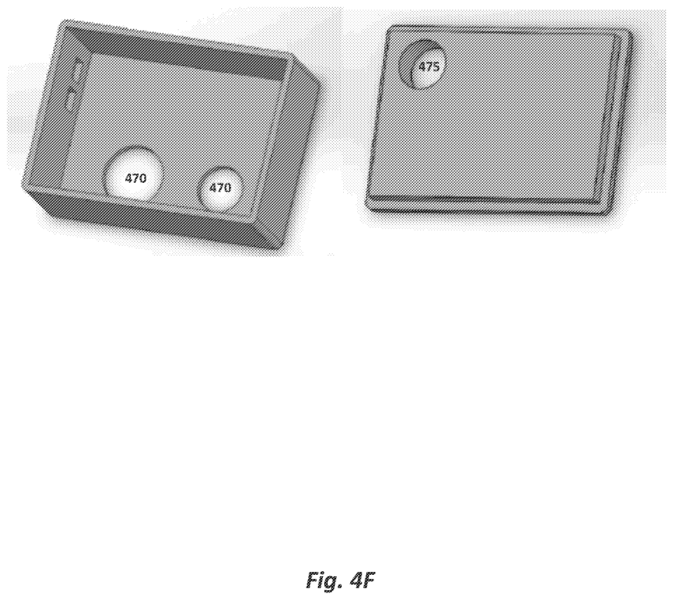

Referring now to FIG. 1, an example system for detecting a motor developmental delay and/or neurodevelopmental disorder of an infant is described. This disclosure contemplates that the infant can be a term infant (i.e., born on or after complete gestation period) or preterm infant (i.e., born before complete gestation period). The system can include one or more sensors 102 and a computing device 104 operably coupled to the sensors 102. As described herein, in some implementations, the sensors 102 are optionally incorporated into an infant sensor suit (described below with respect to FIGS. 4A-4F). Additionally, this disclosure contemplates that the computing device 104 can be the example computing device described with regard to FIG. 3. Alternatively or additionally, an application (e.g., a mobile app) can optionally run on the computing device 104, and the application can receive the data measured by the sensors 102. A user (e.g., clinician and/or parent) can use the application to monitor the system and/or interpret the data. The sensors 102 and the computing device 104 can be connected by a communication link 106. In some implementations, the sensors 102 and the computing device 104 exchange data over a low power wireless link such as using BLUETOOTH wireless technology. Although BLUETOOTH wireless technology is provided as an example, this disclosure contemplates the communication link 106 is any suitable communication link. For example, a communication link may be implemented by any medium that facilitates data exchange between the sensors 102 and the computing device 104 including, but not limited to, wired, wireless and optical links. Example communication links include, but are not limited to, a LAN, a WAN, a MAN, Ethernet, the Internet, or any other wired or wireless link such as WiFi, WiMax, 3G, 4G, or 5G.

The sensors 102 can be configured for placement on limb segments of the infant. For example, the sensors can be configured for placement at the infant's thigh, shin, and/or foot. As described herein, the sensors 102 can be incorporated into the infant's clothing. The sensors 102 can be used to detect motion of the infant's limb segments, e.g., thigh, shin, and/or foot. For example, in some implementations, a sensor 102 is placed at the thigh, shin, and foot of each of the infant's legs (i.e., two total sensors or one sensor per lower limb). In other implementations, a respective sensor 102 is placed at two of the thigh, shin, and/or foot of each of the infant's legs (i.e., four total sensors or two sensors per lower limb). In yet other implementations, a respective sensor 102 is placed at each of the thigh, shin, and foot of each of the infant's legs (i.e., six total sensors or three sensors per lower limb). As described herein, receiving motion data from more than one sensor 102 can improve accuracy of the system. Alternatively or additionally, receiving motion data from a particular sensor 102 may provide sufficient accuracy. For example, motion data received from a sensor positioned at the thigh (e.g., least accurate sensor) may be less important than motion data received from a sensor 102 positioned at the foot (e.g., most accurate sensor). This disclosure contemplates that the sensors 102 can be inertial measurement units (IMU). IMUs are sensors that include one or more accelerometers, gyroscopes, and/or magnetometers. IMUs are capable of measuring linear and angular motion of a body. IMUs are known in the art and are therefore not described in further detail herein. METAWEARC Board from MBIENTLAB, INC. of San Francisco, Calif. is an example IMU.

The computing device 104 can be configured to receive and process motion data detected by the sensors 102. For example, the computing device 104 can receive, from the sensor(s) 102, motion data associated with the infant's gross motor activity. Gross motor activity can include a plurality of spontaneous kicking movements. In other words, the infant's gross motor activity is spontaneous kicking activity. Spontaneous kicking is an early display of gross motor skill, and such kicking can be used to identify abnormal neuromotor function by an infant, which includes detecting neurodevelopmental disorders. It should be understood that analyzing gross motor activity is different than analyzing any specific type(s) of movement.

The computing device 104 can analyze, for example using artificial intelligence (Al), the motion data received from the sensors 102 to detect a kinematic feature. For example, a machine learning algorithm can be used to detect the kinematic feature in the motion data. Machine learning algorithms build a mathematical model based on a training data set, which enables the machine learning algorithm to make predictions without explicit programming. Machine learning algorithms can be supervised learning algorithms (e.g., where the training data set includes inputs and known outputs) or unsupervised learning algorithms (e.g., where the training data set includes only inputs). Machine learning algorithms are known in the art and are therefore not described in further detail below. In examples described herein, the machine learning algorithm can be a thresholding algorithm, a K-nearest neighbors (KNN) algorithm, or a Gaussian mixture model (GMM). It should be understood that these algorithms are provided only as examples and that other types of machine learning algorithms including, but not limited to, neural networks, decision trees, and support vector machines, can be used.

In some implementations, the kinematic feature can be a percentage of time the infant spent in a motion state. Optionally, the motion states can be no motion, unilateral motion, and bilateral motion. The no motion state is when neither one of the infant's lower limbs is spontaneously kicking. Unilateral motion is when only the infant's dominant lower limb is spontaneously kicking. It should be understood that which lower limb is dominant depends on the particular infant. Bilateral motion is when both of the infant's lower legs are spontaneously kicking. Thus, the motion data can be analyzed by the machine learning algorithm to classify the gross motor activity into one of the motion states. Accordingly, the amount of time the infant spends in each motion state during a test period can be determined, and the percentage of time in each motion state can be calculated.

It should be understood that the percentage of time spent in a motion state is only one example kinematic feature. This disclosure contemplates that the kinematic feature is not limited to percentage of time spent in a motion state. For example, the kinematic feature can be one of the features shown in Table I below. This disclosure contemplates that a machine learning algorithm can be used to analyze motion data received from the sensors 102 and detect the kinematic features of Table I. As shown in the table, these kinematic features may correlate with infant age, which provides information that can be useful for detection of neurodevelopmental disorders.

TABLE I

| | | | Relationship to | |
|---|---|---|---|---|
| Kinematic Feature | | Description | Infant Age | Source |
| Kick Frequency | | Number of kicks by | Increases with age | [a] see table 1 for |
| (where kick is defined as a | | each leg (cycles per | for infants with | other clinical |
| flexion then an extension | | minute) | VLBW and low GA | studies using this |
| movement of the lower limb) | | | | metric (or similar |
| | | | | metric) |
| Spatiotemporal | Kick | Excursion of hip | Increase with age | [a], [c] |
| Organization | Amplitude | flexion during a kick | | |
| | | cycle | | |
| | Phase | Duration of each kick | Longer intra-kick | |
| | | phase (flexion, intra- | pause and shorter | |
| | | kick pause, extension, | inter-kick pause | |
| | | and inter-kick pause) | with age | |
| Inter-joint Coordination | | Pair-wise cross- | High in newborn | [a] see table 1 for |
| (also called Intra-limb | | correlations of joint | period | other clinical |
| Coordination) | | angles at the hip, knee | | studies using this |
| | | and ankle | | metric |
| Inter-limb Coordination | | Frequency of | Decreasing | [a] see table 1 for |
| | | reciprocal, unilateral, | percentage of | other clinical |
| | | and synchronous | unilateral kicks | studies using this |
| | | kicking (predominant | and increasing | metric |
| | | coordination pattern | percentage of | [b] called “type |
| | | also noted) | synchronous | of each |
| | | | kicking with age | movement” and |
| | | | | have |
| | | | | asynchronous |
| | | | | movement rather |
| | | | | than reciprocal |
| Phase Lag | | Difference between | Small in newborn | [a] see table 1 for |
| | | time at which joints | period | clinical studies |
| | | started moving or | | using this metric |
| | | reached peak | | |
| | | excursion | | |
| Constrained Movement | | Variance in duration | Constrained in | [a] see table 1 for |
| Duration | | in the | newborn period | clinical studies |
| | | flexion/extension | (invariant | using this metric |
| | | phases | durations in the | |
| | | | flexion and | |
| | | | extension phases) | |
| Duration of Movement | | Number of samples | No change | [b] |
| (Here, movement identified | | within each | reported in this | |
| each time the infant's leg | | movement (could be | work, but in ref 13 | |
| paused or changed direction) | | temporal duration as | this increased | |
| | | well) | with age | |
| Average Acceleration | | Average acceleration | No change | [b] |
| | | for a movement | reported in this | |
| | | | work | |
| Peak Acceleration | | Peak acceleration for | No change | [b] |
| | | a movement | reported in this | |
| | | | work | |
| Joint Angles | | Joint angle positions | Compared | [c], [e], [f], [g] |
| | | at beginning and end | preterm (PT) to | |
| | | of flexion and | full term (FT) | |
| | | extension phase | infants | |
| Joint Angle Excursion | | Magnitude of the joint | Compared PT to | [c], [e], [f], [g] |
| | | excursion within each | FT infants | |
| | | phase (range of | | |
| | | motion) | | |
| Peak Joint Angles | | Maximum angle | Compared PT to | [c], [e], [f], [g] |
| | | positions within each | FT infants | |
| | | phase | | |
| Peak Joint Velocities | | Of each joint during | Compared PT to | [c], [e], [f], [g] |
| | | flexion and extension | FT infants | |
| | | phases | | |
| Intra Limb | Discrete | Difference of joint | Compared PT to | [c], [e], [f], [g] |
| Coordination | Relative | angles at the end of | FT infants | |
| | Phase | flexion phase, | | |
| | | beginning of | | |
| | | extension phase, and | | |
| | | flexion/extension | | |
| | | peak velocities (points | | |
| | | of potential need for | | |
| | | control) (i.e. high | | |
| | | velocity or joint | | |
| | | changes) where 0 | | |
| | | means in phase, pi | | |
| | | indicates out of phase | | |
| | Relation of | Correlation between | Compared PT to | [c], [e], [f], [g] |
| | Joint Angle | amplitude of the | FT infants | |
| | Amplitude to | movement phases | | |
| | Peak Velocity | and their peak | | |
| | | velocities\ | | |

Sources:
[a] Jerig, Suh-Fang, Li-Chiou Chen, and Kuo-Inn Tsou Yau. “Kinematic analysis of kicking movements in preterm infants with very low birth weight and full-term infants.” Physical therapy 82.2 (2002): 148-159.
[b] Trujillo-Priego, Ivan A., and Beth A. Smith. “Kinematic characteristics of infant leg movements produced across a full day.” Journal of rehabilitation and assistive technologies engineering 4 (2017): 2055668317717461.
[c] Fetters, Linda, et al. “Kicking coordination captures differences between full-term and premature infants with white matter disorder.” Human movement science 22.6 (2004): 729-748.
[d] Landgraf, Jocelene de Fátima, Raquel de Paula Carvalho, and Eloisa Tudella. “Method for the kinematic analysis of kicking movement in infants.” Fisioterapia e Pesquisa 20.1 (2013): 56-63.
[e] Chen, Y., L. Fetters, K. G. Holt, and E. Saltzman. “Making the mobile move: Constraining task and environment.” Infant Behavior and Development 25 (2002): 195-220.
[f] Jeng, S.-F., L.-C. Chen, and K.-I. T. Yau (2002). “Kinematic analysis of kicking movements in preterm infants with very low birth weight and full-term infants.” Physical Therapy 82 (2002): 148-159.
[g] Jensen, J. L., B. D. Ulrich, E. Thelen, K. Schneider, and R. F. Zernicke “Adaptive dynamics of the leg movement patterns of human infants: I. The effects of posture on spontaneous kicking.” Journal of Motor Behavior 26 (1994): 303-312.

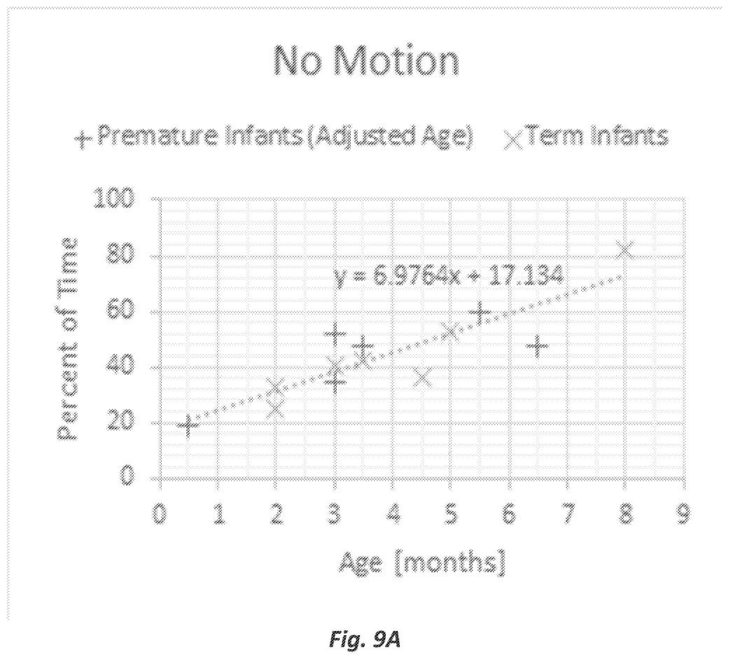

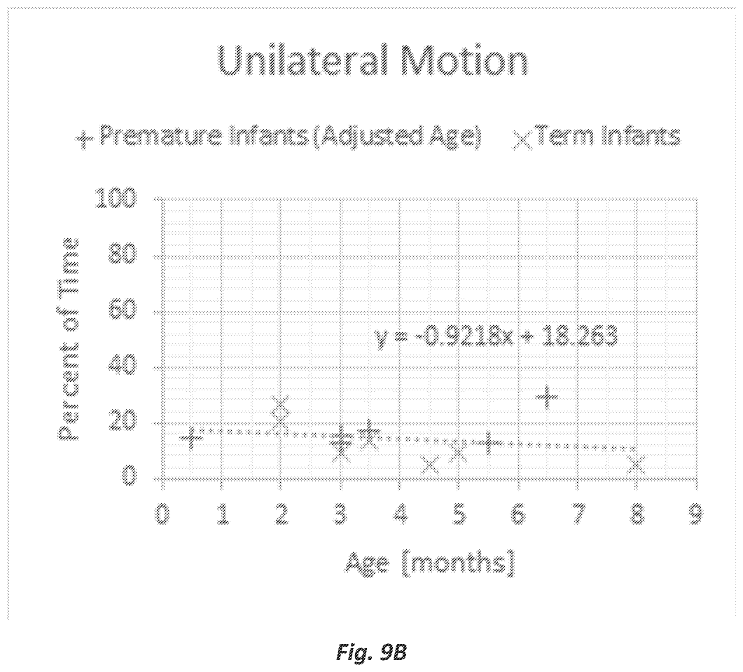

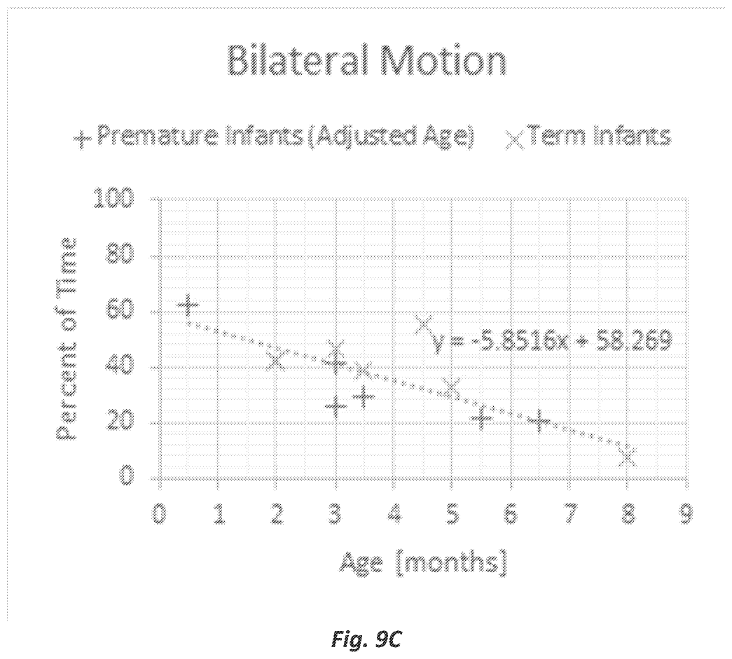

After detecting the kinematic features, the computing device 104 can compare the kinematic feature to an expected relationship between the kinematic feature and infant age. A kinematic feature can have an expected relationship with infant age. This disclosure contemplates that infant age can be birth age or adjusted age, where adjusted age is birth age adjusted for preterm infants to account for birth prior to due date. For example, as shown in FIGS. 9A-9C (adjusted age) and 10A-10C (birth age), the percentage of time in particular motion states (e.g., no motion, unilateral motion, bilateral motion) have expected relationships with infant age. Rest (i.e. no motion state) increase with infant age, while unilateral and bilateral motion decrease with infant age. Bilateral motion decreases at a more rapid rate than unilateral motion as infant age increases. This disclosure contemplates that one or more kinematic features can be compared to their respective expected relationships.

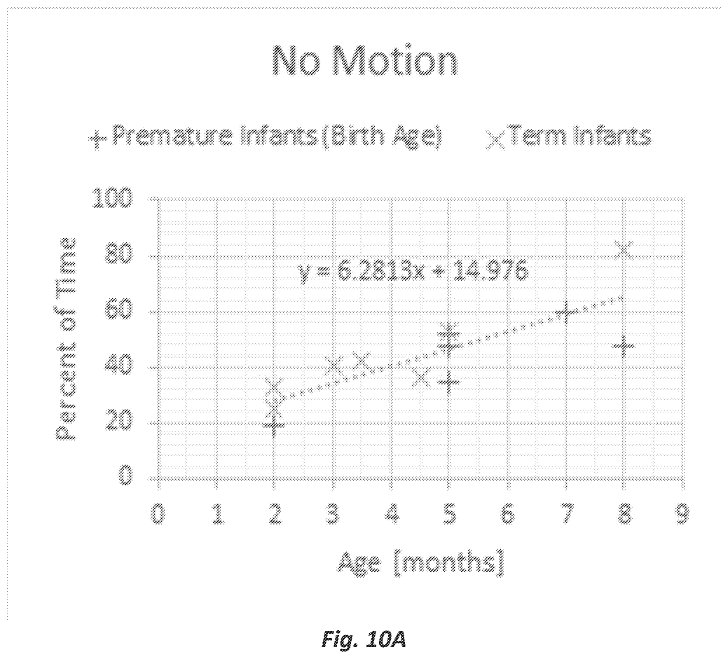

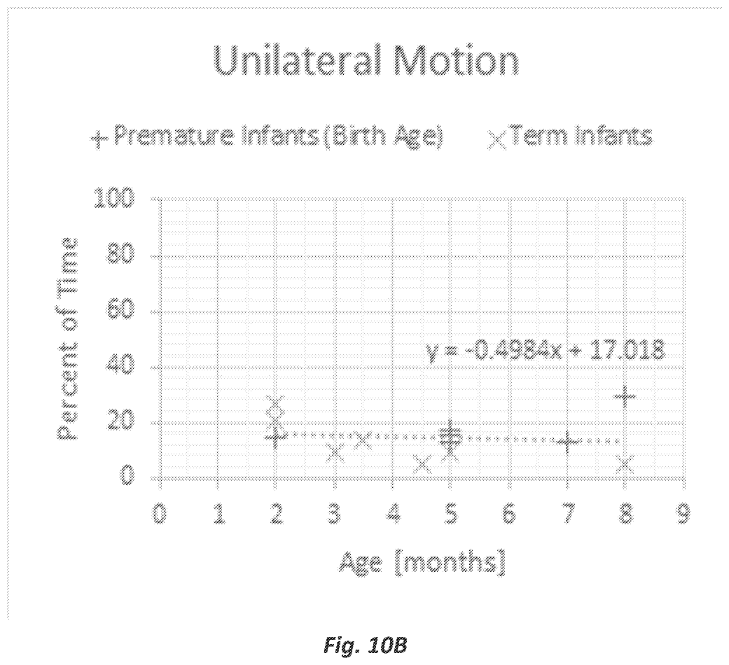

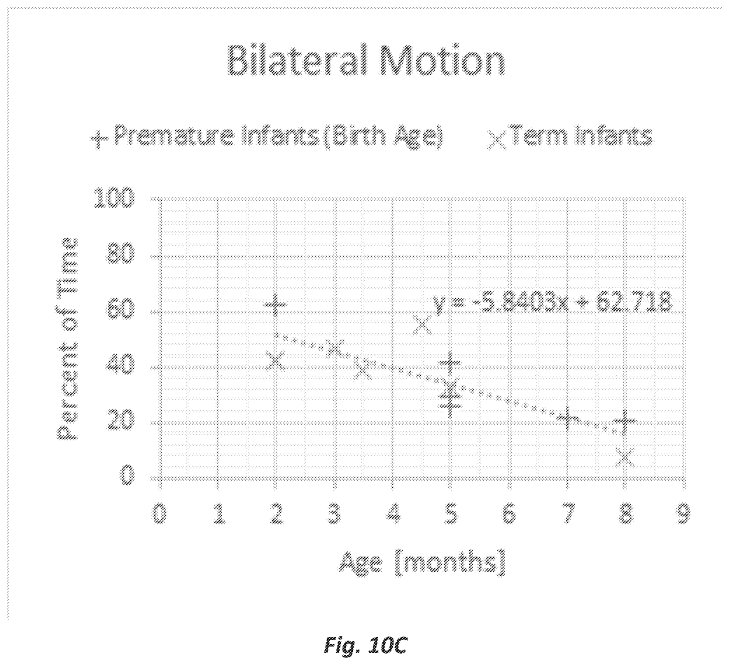

The computing device 104 can detect the neurodevelopmental disorder based on the comparison. As described above, for a given infant age, the infant is expected to exhibit an expected kinematic feature. For example, for a given infant age, the infant is expected to exhibit a specified percentage of time in the no motion state (and/or unilateral motion state and/or bilateral motion state). The respective percentage of time spent in any one or more of the motion states (e.g., no motion, unilateral, bilateral) provides information that can be used to detect neurodevelopmental disorder. For example, if the infant exhibits no motion, unilateral, and/or bilateral motion at a % value different than expected for the infant's age, then the infant is motor developmentally delayed for the infant's age. If the infant exhibits no motion at a % value less than expected for the infant's age, then the infant is motor developmentally delayed (see FIGS. 9A and 10A). Alternatively or additionally, if the infant exhibits unilateral motion at a % value more than expected for the infant's age, then the infant is motor developmentally delayed (see FIGS. 9B and 10B). Alternatively or additionally, if the infant exhibits bilateral motion at a % value more than expected for the infant's age, then the infant is motor developmentally delayed (see FIGS. 9C and 10C). This can provide an indication of a neurodevelopmental disorder such as cerebral palsy. It should be understood that cerebral palsy is only provided as an example neurodevelopmental disorder. This disclosure contemplates that the neurodevelopmental disorder can be a condition in which development of the central nervous system is disturbed other than cerebral palsy. Additionally, this disclosure contemplates that the computing device 104 can detect a motor developmental delay based on the comparison. For example, if the infant exhibits no motion, unilateral, and/or bilateral motion at a % value different than expected for the infant's age, then the infant is motor developmentally delayed for the infant's age. If the infant exhibits no motion at a % value less than expected for the infant's age, then the infant is motor developmentally delayed (see FIGS. 9A and 10A). Alternatively or additionally, if the infant exhibits unilateral motion at a % value more than expected for the infant's age, then the infant is motor developmentally delayed (see FIGS. 9B and 10B). Alternatively or additionally, if the infant exhibits bilateral motion at a % value more than expected for the infant's age, then the infant is motor developmentally delayed (see FIGS. 9C and 10C).

### Example Methods

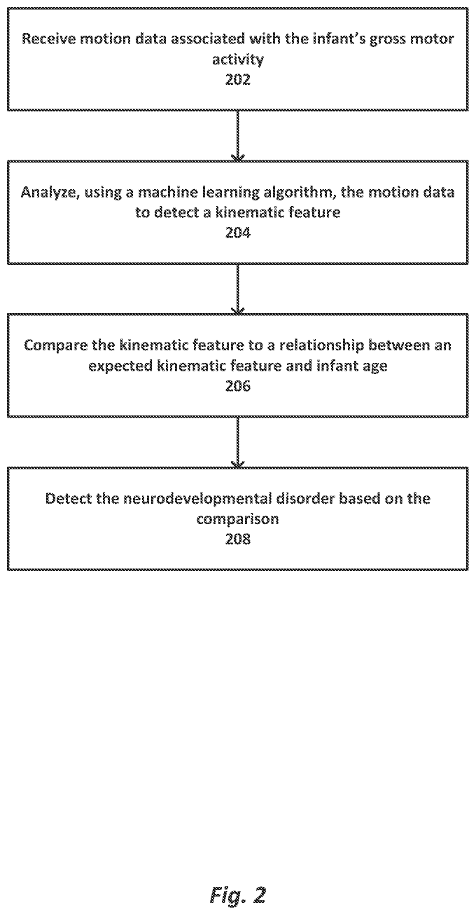

Referring now to FIG. 2, example operations for detecting a neurodevelopmental disorder of an infant are described. This disclosure contemplates that the system shown in FIG. 1 (e.g., sensors 102, computing device 104) can be used to implement the example operations. At step 202, motion data associated with the infant's gross motor activity is received. At step 204, the motion data is analyzed, using a machine learning algorithm, to detect a kinematic feature. At step 206, the kinematic feature is compared to an expected relationship between the kinematic feature and infant age. At step 208, the neurodevelopmental disorder is detected based on the comparison.

Alternatively or additionally, example operations for detecting a motor developmental delay of an infant are described. This disclosure contemplates that the system shown in FIG. 1 (e.g., sensors 102, computing device 104) can be used to implement the example operations. Motion data associated with the infant's gross motor activity can be received. The motion data can be analyzed, using a machine learning algorithm, to detect a kinematic feature. The kinematic feature can be compared to an expected relationship between the kinematic feature and infant age. The motor developmental delay can be detected based on the comparison. For example, if the infant exhibits no motion, unilateral, and/or bilateral motion at a % value different than expected for the infant's age, then the infant is motor developmentally delayed for the infant's age. If the infant exhibits no motion at a % value less than expected for the infant's age, then the infant is motor developmentally delayed (see FIGS. 9A and 10A). Alternatively or additionally, if the infant exhibits unilateral motion at a % value more than expected for the infant's age, then the infant is motor developmentally delayed (see FIGS. 9B and 10B). Alternatively or additionally, if the infant exhibits bilateral motion at a % value more than expected for the infant's age, then the infant is motor developmentally delayed (see FIGS. 9C and 10C).

It should be appreciated that the logical operations described herein with respect to the various figures may be implemented (1) as a sequence of computer implemented acts or program modules (i.e., software) running on a computing device (e.g., the computing device described in FIG. 3), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

### Example Computing Device

Referring to FIG. 3, an example computing device 300 upon which the methods described herein may be implemented is illustrated. It should be understood that the example computing device 300 is only one example of a suitable computing environment upon which the methods described herein may be implemented. Optionally, the computing device 300 can be a well-known computing system including, but not limited to, personal computers, tablets, smartphones, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data transmission medium, to perform various tasks. In the distributed computing environment, the program modules, applications, and other data may be stored on local and/or remote computer storage media.

In its most basic configuration, computing device 300 typically includes at least one processing unit 306 and system memory 304. Depending on the exact configuration and type of computing device, system memory 304 may be volatile (such as random access memory (RAM)), non-volatile (such as read-only memory (ROM), flash memory, etc.), or some combination of the two. This most basic configuration is illustrated in FIG. 3 by dashed line 302. The processing unit 306 may be a standard programmable processor that performs arithmetic and logic operations necessary for operation of the computing device 300. The computing device 300 may also include a bus or other communication mechanism for communicating information among various components of the computing device 300.

Computing device 300 may have additional features/functionality. For example, computing device 300 may include additional storage such as removable storage 308 and non-removable storage 310 including, but not limited to, magnetic or optical disks or tapes. Computing device 300 may also contain network connection(s) 316 that allow the device to communicate with other devices. Computing device 300 may also have input device(s) 314 such as a keyboard, mouse, touch screen, etc. Output device(s) 312 such as a display, speakers, printer, etc. may also be included. The additional devices may be connected to the bus in order to facilitate communication of data among the components of the computing device 300. All these devices are well known in the art and need not be discussed at length here.

The processing unit 306 may be configured to execute program code encoded in tangible, computer-readable media. Tangible, computer-readable media refers to any media that is capable of providing data that causes the computing device 300 (i.e., a machine) to operate in a particular fashion. Various computer-readable media may be utilized to provide instructions to the processing unit 306 for execution. Example tangible, computer-readable media may include, but is not limited to, volatile media, non-volatile media, removable media and non-removable media implemented in any method or technology for storage of information such as computer readable instructions, data structures, program modules or other data. System memory 304, removable storage 308, and non-removable storage 310 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 306 may execute program code stored in the system memory 304. For example, the bus may carry data to the system memory 304, from which the processing unit 306 receives and executes instructions. The data received by the system memory 304 may optionally be stored on the removable storage 308 or the non-removable storage 310 before or after execution by the processing unit 306.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### Example Infant Sensor Suit

The example infant sensor suit described herein allows for the collection of infant kinematic kicking data outside of a clinical setting. This provides a more accessible option for long term collection of infant kicking data. Optionally, this data can be analyzed offline (e.g., as described above with regard to FIG. 2) to detect potential motor development delays. Using this information, clinicians can identify and provide therapy for infants displaying signs of motor development delay. The systems and methods described herein are particularly useful to monitor infants who were born preterm who are at a higher risk for motor development delay.

The infant sensor suit described herein is designed to wearable, comfortable and lightweight. The infant sensor suit exists as two parts: a soft circuit and hard circuit components. The soft circuit (infant suit base) includes conductive thread, conductive fabric, and component connectors. The hard circuit components include sensors, a power source (one or more batteries), and voltage regulators. The infant sensor suit is designed so that the hard circuit components can be easily installed and removed from the infant suit base (soft circuit). This allows for the transfer of the more expensive hard circuit components between different infant suit bases. As such, a single set of hard circuit components can be used with multiple infant suit bases. For example, the same set of hard circuit components can be used as the infant ages. Finally, when the hard circuit components are removed, the infant suit base is machine washable.

Referring now to FIGS. 4A-4D, an example infant sensor suit is described. The infant sensor suit can include an article of clothing 400; a plurality of sensors 405a, 405b, 405c, 405d, 405e, 405f (referred to herein individually and collectively as “sensor(s) 405”); a power source 410 operably coupled to the sensors 405; and a wireless transmitter operably coupled to the sensors 405. The sensors 405, power source 410, and wireless transmitter can be incorporated into the article of clothing 400 as shown in FIGS. 4A-4D. In FIGS. 4A-4D, the article of clothing 400 is infant pants, and the sensors 405 are IMUs (e.g., MetaWear C sensors (from MbientLab), which include wireless transmitters). This disclosure contemplates that the wireless transmitter can be part of the sensor hardware or provided as a separate component. MetaWear C sensors transmit sensor data using the Bluetooth protocol.

The infant sensor suit shown in FIG. 4A includes six MetaWear C sensors (from MbientLab) on each leg of the infant sensor suit 400. Sensors 405a and 405d are configured for placement at the infant's left and right thighs. These sensors are configured to detect motion of the infant's thigh segment. Sensors 405b and 405e are configured for placement at the infant's left and right shins. These sensors are configured to detect motion of the infant's shin segment. Sensors 405c and 405f are configured for placement at the infant's left and right feet. These sensors are configured to detect motion of the infant's foot segment. It should be understood that the number and/or arrangement of the sensors 405 in FIG. 4A are provided only as examples. This disclosure contemplates using different numbers and/or arrangements of sensors 405.

Each of the sensors 405 measures and transmits gyroscopic and accelerometer data at a rate of 100 Hz over a wireless communication link (e.g., using Bluetooth protocol). As described above, the motion data can be transmitted to a remote computing device such as computing device 104 of FIG. 1. In some implementations, the data can be received at an application (e.g., mobile app) that compiles the data together for users. Referring again to FIGS. 4A-4D, the infant sensor suit can be powered by a power source 410. The power source 410 can be located in the back-thigh area of the infant to provide enough cushion to ensure the infant is not poked. In FIG. 4A, the power source 410 is two lithium ion 3.7 V batteries, i.e., one power source 410 per leg of the article of clothing 400. It should be understood that the power source 410 can be a rechargeable battery. In this implementation, sensors 405a-405c are operably connected to one power source 410, while sensors 405d-405f are operably connected to another power source 410. MetaWear C sensors each require 3V. Thus, sensors 405a-405c are connected in parallel with each other, and the voltage source (e.g., power source 410-3.7 V battery) is connected to a RB-Pol-221 (Pololu) voltage regulator that brings the voltage down to 3V. The Pololu voltage regulator produces an adjustable output between about 2.5V and 8V from input voltages between about 2.7V and 11.8V. For the infant sensor suit, the voltage regulator is adjusted to provide output of about 3V from an input of about 3.7V from the lithium ion battery. Additionally, sensors 405d-405f are connected in parallel with each other, and the voltage source (e.g., power source 410-3.7 V battery) is connected to a RB-Pol-221 (Pololu) voltage regulator that brings the voltage down to 3V. In other words, on each leg of the article of clothing 400, there is one 3.7 lithium ion battery (placed in a pocket of the back thigh area of the suit), three MetaWear C sensors, and one voltage regulator. By using a power source/voltage regulator with multiple sensors per leg, it is possible to prolong battery life without loss of measurement capability over time. The power source 410, sensors 405, and voltage regulator are referred to herein as the “hard circuit components”. It should be understood that the voltages ranges above are provided only as examples. The voltages ranges are dependent on the hardware and may be different than those provided as examples.

Additionally, the infant sensor suit can further include a wearable circuit 420 that is incorporated into the article of clothing 400. The wearable circuit 420 can be configured to operably couple the sensors 405 and the power source 410. Optionally, the wearable circuit 420 can include a conductive thread 420a or conductive fabric 420b. The conductive thread 420a can act as “wires” and the conductive fabric 420 can act as “nodes” in the wearable circuit 420. For example, as shown in FIGS. 4C and 4D, connections between the voltage source and sensors can be provided by patches of conductive fabric 420b and conductive thread 420a. The wearable circuit 420 can therefore operably couple the sensors 405 and power source 410 on each leg of the article of clothing 400 shown in FIG. 4A. Different conductive thread gives off different resistances. The resistance is determined by the amount of ply within the conductive thread 420a. The more ply on the thread, the less resistance. Conductive fabric 420b helps disperse current across the entire set of fabric. For the purposes of the suit, the conductive fabric 420b is cut into squares to act as nodes in the wearable circuit 420. Syscom Advanced Materials Liberator 40 Silver is an example conductive thread, and Adafruit Knit Jersey conductive fabric is an example conductive fabric. It should be understood that these are only provided as example conductive thread and fabric and that others can be used in the infant sensor suit. In terms of the creation of a parallel circuit on each leg of the article of clothing 400, the conductive fabric 420b (e.g., patches measuring 2.5 cm×2 cm) acts as nodes within the wearable circuit 420, while the conductive thread 420a acts as the lines that current passes through. Resistance of the conductive thread varies based on length, but the maximum resistance the conductive thread can be limited, for example to about 5 ohms. In this way, when put in conjunction with the conductive fabric, the maximum resistance of the wearable circuit 420 can be about 10 ohms (before being connected to the MetaWear C sensors). In addition, smaller patches of conductive fabric (e.g., patches measuring 1.5 cm×1.5 cm) can be placed at the connection points of where the MetaWear C sensors are arranged. Each of these patches can act as terminals for the sensors to go on. Electrical terminals (e.g., 9V connectors terminals) can be attached to the conductive fabric via small screw and nuts. Positive terminals are placed on one side (e.g., right or left) of the leg, while negative terminals are placed on the other side (e.g., left or right) of the leg. In addition, fabric can be placed under these terminals to prevent potential harm to the infant. The connection between the conductive fabric and conductive thread can be sewed in a way to ensure a secure connection by applying a zig-zag stitch to the conductive fabric with the conductive thread three times. The wearable circuit 420 (conductive fabric and thread) is referred herein to as the “soft circuit”.

Alternatively or additionally, as shown in FIG. 4D, the infant sensor suit can further include a sensor pocket 430 that is attached to the article of clothing 400. This disclosure contemplates that one or more sensor pockets 430 can be provided. For example, a sensor pocket can be provided in the vicinity of each of the thigh region, shin region, and foot region of the infant sensor suit. In other implementations, a sensor pocket 430 that spans the length of the leg of the suit can be provided. The sensor pocket 430 can be configured to receive the sensors, voltage regulators, and/or power source. Optionally, a respective position of one or more of the sensors, voltage regulators, and/or power source are configured to be adjustable within the sensor pocket 430. This facilitates repositioning of one or more of the MetaWear C sensors relative to the infant's anatomy. The sensor pocket 430 assists to cover the MetaWear C sensors and also help in moving the MetaWear C sensors up and down the infant's legs. For example, by using a double-sided zipper and/or hook-and-loop fastener, the MetaWear C sensor is connected to the bottom zipper by using a 3D printed peg that attaches to the sensor container (described below). Once the MetaWear C sensor is connected to the sensor pocket 430, the leads can be connected power the MetaWear C sensor. To adjust the necessary location of the MetaWear C sensor, the bottom zipper is moved up and down. The bottom of the container has hook-and-loop fasteners that attach to the fabric to ensure the sensor will stay in place. The top zipper acts as a lock to keep everything in place.

As described herein, one or more of the sensors can be repositioned within the sensor pocket 430 using zippers and/or hook-and-loop fasteners (e.g., VELCRO fasteners from VELCRO INDUSTRIES N.V. of the United Kingdom) or zippers. For example, the MetaWear C sensors are placed in the thigh, shin, and foot area of each leg of the infant sensor suit. The sensor pockets that house the batteries can be made of fire retardant fabric as a safety precaution. Each MetaWear C sensor can be operably coupled (e.g., soldered) to an electrical connector (e.g., 9V connector), so that MetaWear C sensors can snap easily with the infant suit. In addition, each MetaWear C sensor can optionally be placed into a container (e.g., a 3D printed container). An example sensor container is shown in FIG. 4E. The sensor container is designed so that the MetaWear C sensor is always be oriented in one direction. The sensor container has three elements. The base 450 houses the MetaWear C sensor. The cap 455 contains pegs on the bottom that fit into the positive and negative cell terminals. This is to make sure the MetaWear C sensor is secure when housed in the base container and is not moving left and right. The top of the cap contains a square hole 460 which connects to the third part of the sensor container, i.e., the zipper hook. The zipper hook attaches to the bottom zipper of the jacket. This allows the user to attach the sensor container to the zipper and move the container up and down. The sensor container has a hook-and-loop connector at the bottom that solidifies placement of the MetaWear C sensor. A similar setup is done for the infant's foot except its sensor container does not have a slot for the zipper hook as the zipper hook is not needed.

Similarly, the voltage regulator can also be placed in its own container. An example regulator container is shown in FIG. 4F. The regulator container has two holes 470 on the bottom meant for the connection terminals that connect to the suit. The 9V connector goes through the holes and is connected to the voltage regulator. The regulator container houses both the 9V connectors and the voltage regulator. The sides of the regulator container contain square holes to connect the battery input. The cap for the regulator container contains a hole 475 in the corner that is for the use of moving the potentiometer to adjust the regulator output voltage. Should it be needed, the potentiometer can be turned while the regulator is still in the box. The hole can be covered with a bumper.

In some implementations, the MetaWear C sensors in the thigh and shin areas of the infant sensor suit can be attached to a double-sided zipper provided in the sensor pocket 430. Additionally, hook-and-loop connections can be provided on the base of the MetaWear C sensor containers as well as on the area where they will be placed on the suit. The double-sided zipper can be used to control the location of the MetaWear C sensor on the suit, as infant size varies even when using the same size infant pant. Alternatively or additionally, the MetaWear C sensor arranged near the infant's foot can be attached to a detachable sock that the sensor is velcroed onto, which is then covered by another sock for added padding and comfort. This disclosure contemplates that any conductive elements on the surface of the suit, aside from the terminals housing the electrical connectors, can be covered by placing fusible interfacing on a secondary fabric that glues on to the infant sensor suit. By combining the soft circuit and hard circuit components on each leg of the infant sensor suit, gyroscopic and accelerometer data can be used to transmit kinematic kicking data to a remote computing device for further analysis.

### EXAMPLES

### Example 1

As described above, spontaneous kicking in infants is one of the earliest displays of motor skills. Abnormalities observed in these displays are an important indicator of later abnormal neuromotor function. However, these abnormalities are not well defined and difficult to detect outside of direct clinical observation. The systems and methods described in Example 1 facilitate analysis of spontaneous kicking in infants. For example, to allow for extended, non-clinical observation of spontaneous kicking, IMU sensors are attached to the limb segments of the infant's legs. An activity detection algorithm is then used to quantify kicking activity derived from collected measurement data. Example 1 describes the technique in detail and discusses results from kicking data acquired from term and low-risk preterm infants.

Introduction

Spontaneous kicking is one of the earliest displays of motor skills and is an important precursor to later voluntary motor control [6]. Thus, abnormal neuromotor function displayed through spontaneous kicking indicates later abnormal neuromotor function [7-8]. However, abnormalities in spontaneous movements are not well defined and are not readily observable through functional motor milestone assessments.

Example 1 describes an in-home system for the early detection of CP that will allow for the extended observation of infant spontaneous kicking outside of the clinical setting. Sensors attached to the limb segments of the infant's legs enable one to gather infant kicking kinematic data over long periods of time. From this data, kicking kinematics can be computed and various features of infant spontaneous kicking can be determined

Related Work

Numerous techniques have been developed to determine characteristics of typical and atypical infant motor development. In [10], motion tracking systems were used to gather infant pose data and track infant movement. Karch et al. used electromagnetic tracking to examine upper and lower limb motions in infants of three months adjusted age [10]. These methods provided precise spatial tracking of infant spontaneous movement. However, such approaches and others like them require specialized equipment that is unsuitable for use outside of a laboratory or clinical setting.

Other approaches utilize optical devices such as video or depth cameras to analyze infant motion data. In [11], kinematic analysis of video data with joint markers on the infants' lower limbs is used to establish baseline data of early spontaneous movement in preterm infants. Other approaches attempt to identify at risk infants using marker-less video data [12]. In [13-14], depth cameras are used to capture infant movement and estimate the infant's pose without the need for markers or sensors attached to the infant. However, these methods often make assumptions about the position of the joints and require a specific configuration between the camera and the infant being filmed, making these approaches non-ideal for in-home usage.

Still other approaches use wearable technology or clothing with embedded sensors. In [15], accelerometer data is used to identify motor milestones in infants and identify at risk infants. Smith et al. gather infant kicking data over the course of a full day to determine the daily quantity of infant leg movement to predict walking onset [16]. Due to their low cost and ability to be utilized in multiple settings, these approaches are suitable for in-home usage. However, these approaches are somewhat limited as they require a strict definition of what movements qualify as a kick.

Methods

According to the systems and methods of Example 1, to enable the early detection of neurodevelopmental motor disorders among children, the system allows for a longer observation time than typically allowed in a clinical setting and is adaptable to variations in infant size and age. The system uses an infant sensor suit to gather motion data associated with an infant's spontaneous kicking patterns. Collected data is then analyzed to determine instances of kicking activity as the first measure for calculating infant kicking kinematic data over long periods of time.

Infant Sensor Suit and Data Collection App

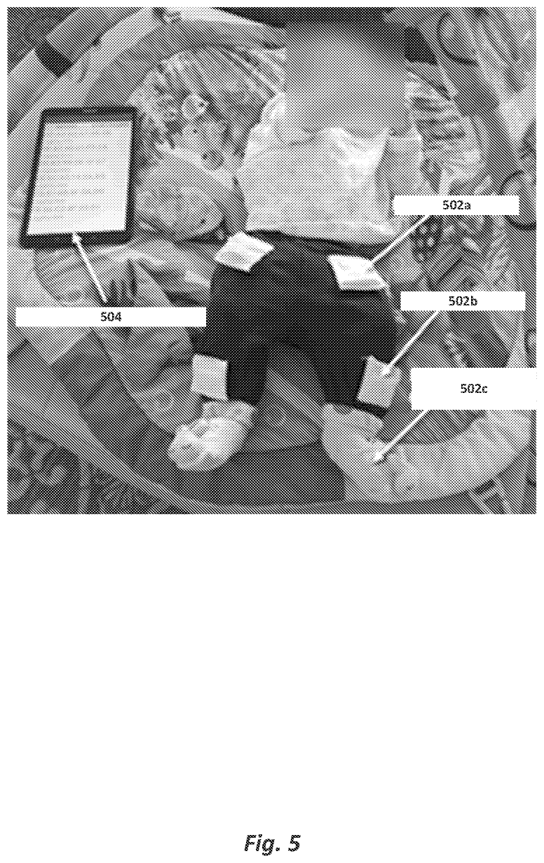

To enable ease-of-collection in the home, the system couples a Bluetooth-connected infant sensor suit with a data collection app, resident on a mobile device. The infant sensor suit pairs a one-piece infant suit and rattle socks with six 6-axis IMU sensors powered by a coin cell battery (MbientLab's MetawearC). As shown in FIG. 5, the suit incorporates 3 sensors per leg (i.e., sensors 502a, 502b, 502c), placed on the thigh, shin, and foot to gather 3-axis acceleration and gyroscope data for each of the limb segments. In FIG. 5, the sensors on the left leg of the infant sensor suit are labeled. It should be understood that sensors are also placed on the right leg of the infant sensor suit. These sensors utilize Bluetooth Low Energy (BLE) technology for data transfer to a custom app, which is running on a computing device 504. The app allows clinicians or parents to gather data from the sensor suit while monitoring battery life and connectivity of each sensor.

Data Collection Procedure

Eight infants, three males and five females, aged between 2 and 11 months adjusted age were observed for this study (Table II). Six of the eight infants were born full term while the remaining two infants were born premature, but considered low risk. Infants are placed supine on a flat, padded surface while wearing the infant sensor suit. The infant's legs are held stationary at the beginning of each segment of data. Then the infant is encouraged to kick through verbal stimulation in this position until it is determined that the infant needs to rest, not exceeding 5-minute segments. Several segments of data are collected with each session yielding up to 20 minutes of kicking data. Each data collection segment is also filmed using a video timestamping application.

Kicking data was collected for both the left and the right leg of six of the infants. Kicking data for only the left leg was collected for two of the infants, A1 and A2. Session length varied between infants due to infant emotional state and averaged to 11 minutes.

Activity Detection Algorithm

During kicking, acceleration and angular rate data were collected at a sampling rate of 100 Hz. To account for local dependencies between data points, a sliding window was used with a window width of 250 milliseconds (ms) and step of 100 ms for each segment of data. These values were determined through previous experiments to optimize the accuracy of kicking detection while compensating for sensor noise. A Stance Hypothesis Optimal Detector (SHOD) was used to determine instances of gross motor activity for each leg. SHOD is a magnitude based method that uses both acceleration and angular rate data to increase the precision and accuracy of an activity detector [17]. The figure of merit V is used as the input of the activity detector and is computed as follows.

$$V ( n ) = \frac{1}{N} {\sum }_{k = 1}^{N} ( \frac{1}{{σ}_{a}^{2}} {‖ {a}_{k} - g \frac{{\overline{a}}_{n}}{‖ {\overline{a}}_{n} ‖} ‖}^{2} + \frac{1}{{σ}_{ω}^{2}} {‖ {ω}_{k} ‖}^{2} ) < γ ,$$

where: n refers to a specific frame which represents the content (acceleration and angular rate data) of a sliding window; aₖ and ωₖ are the acceleration and angular rate vector respectively for observation k of a specific frame n; āₙ is the mean of the acceleration vector of a specific frame n; g is the magnitude of acceleration due to gravity (1 g).

TABLE II
Infant Demographics

| | | | Age at | Gestation |
|---|---|---|---|---|
| | Session | | Testing | Age |
| Infant^{a} | Length | Sex | (months) | (weeks) |
| A₁ | 1 m 28 s | M | 11 | 38 |
| A₂ | 4 m 32 s | M | 11 | 38 |
| B | 14 m 56 s | F | 3 | 38 |
| C₁ | 14 m 39 s | F | 5 | 32 |
| C₂ | 5 m 21 s | M | 5 | 32 |
| D | 20 m 41 s | F | 4.5 | 38 |
| E | 10 m 57 s | F | 2 | 38 |
| F | 15 m 23 s | F | 2 | 39 |

^{a}Subscripts denote multiple births

The constants determined are as follows: N=25 is the number of samples in a frame determined by the desired length of the frame in seconds multiplied by the sampling frequency; σₐ²=3.24×10⁻⁶, variance of the acceleration signal noise, and σ_{ω}²=0.0049, variance of the angular rate signal noise, are determined from specifications of the IMU; y is the threshold that characterizes the decision of the detector.

In this Example, γ is determined using a leave-one-out approach. The leave-one-out approach is a method in which the threshold value associated with the maximum efficiency is determined for each of the remaining segments of data from a receiver operating characteristic (ROC) plot. The point of maximum efficiency represents the cutoff that maximizes both sensitivity and specificity. These values are then averaged to determine y for the left-out segment.

The figure of merit, V, is determined for each of the sensors per leg to calculate instances of kicking activity. The identified periods of activity associated with individual sensors are then combined to classify overall activity for the leg. Two decision fusion methods for combining sensor data were considered. The first method (OR) classified a kicking activity as active if a positive instance of leg activity was detected by any one sensor. The second method (MODE) classified active kicking activity if two or more sensors detected leg activity.

Construction of Truth

Instances of leg motion activity in the timestamped videos were codified independently by three coders. Each coder was asked to identify moments of time when the infant's leg was moving. The results were resampled to match the sampling frequency of the algorithm and then used to construct the truth vector of identified motion activity. If there was disagreement in motion activity between the coders, the most popular identifier (2 out of 3) at that timestamp was used.

Results

Table III displays the average detector performance when using the OR decision fusion method. For this method, the overall average accuracy was 77% for the left leg and 78% for the right leg. Table IV displays the average detector performance when using the MODE decision fusion method. For the MODE decision method, the overall average accuracy for both the left and right leg was 78%. Additionally, the average sensitivity decreased and the average specificity increased for both legs when compared to the OR decision fusion method.

TABLE III
Performance of Detector OR Method

| | Left Leg | | | Right Leg | | |
|---|---|---|---|---|---|---|
| Infant | Acc. | Spec. | Sens. | Acc. | Spec. | Sens. |
| A₁ | 0.73 | 0.66 | 0.90 | — | — | — |
| A₂ | 0.78 | 0.73 | 0.92 | — | — | — |
| B | 0.82 | 0.76 | 0.87 | 0.80 | 0.71 | 0.87 |
| C₁ | 0.72 | 0.63 | 0.80 | 0.72 | 0.65 | 0.74 |
| C₂ | 0.72 | 0.69 | 0.78 | 0.73 | 0.70 | 0.78 |
| D | 0.85 | 0.80 | 0.91 | 0.85 | 0.80 | 0.91 |
| E | 0.74 | 0.72 | 0.79 | 0.78 | 0.74 | 0.80 |
| F | 0.81 | 0.75 | 0.85 | 0.81 | 0.75 | 0.88 |
| AVERAGE | 0.77 | 0.72 | 0.85 | 0.78 | 0.73 | 0.83 |

TABLE IV
Performance of Detector MODE Method

| | Left Leg | | | Right Leg | | |
|---|---|---|---|---|---|---|
| Infant | Acc. | Spec. | Sens. | Acc. | Spec. | Sens. |
| A₁ | 0.78 | 0.73 | 0.86 | — | — | — |
| A₂ | 0.80 | 0.80 | 0.84 | — | — | — |
| B | 0.83 | 0.81 | 0.81 | 0.81 | 0.76 | 0.82 |
| C₁ | 0.73 | 0.71 | 0.77 | 0.67 | 0.69 | 0.69 |
| C₂ | 0.71 | 0.72 | 0.74 | 0.73 | 0.73 | 0.73 |
| D | 0.86 | 0.85 | 0.86 | 0.86 | 0.84 | 0.88 |
| E | 0.74 | 0.76 | 0.75 | 0.78 | 0.78 | 0.70 |
| F | 0.81 | 0.80 | 0.81 | 0.82 | 0.80 | 0.83 |
| AVERAGE | 0.78 | 0.77 | 0.81 | 0.78 | 0.77 | 0.78 |

Overall, infant D had the highest average accuracy among the subjects for both methods while infant C1 had the lowest average accuracy. The OR method tended to overestimate periods of activity resulting in many false positives and low specificity. The MODE method tended to reduce the number of false positives and increased the specificity of the detector as a result. However, the MODE method also increased the number of false negatives, subsequently leading to a decrease in sensitivity as compared to the OR method.

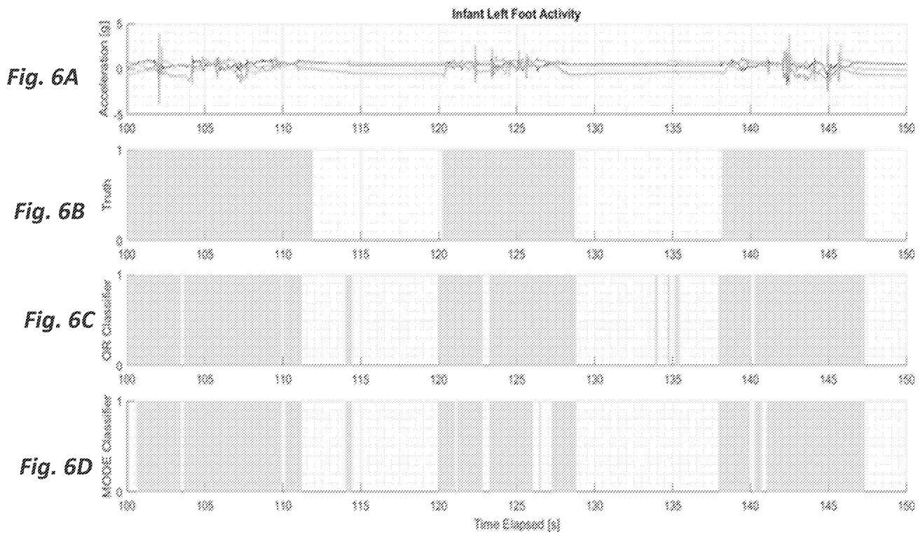

FIGS. 6A-6D use a sample of left foot data from infant D to illustrate these points. The OR (OR classifier v. time plot) method falsely identified periods of activity as compared to ground truth (truth v. time plot) from 110-120s and from 130-135s. The MODE method (MODE classifier v. time plot) did not yield as many instances of false positives (i.e. 130-135s). But, the MODE method did not correctly identify periods of activity, such as from 120-129s, that were correctly identified using the OR method.

### Conclusion

In this Example, periods of activity from infant kicking data were identified using two decision fusion methods to determine the overall activity for the leg: an OR method and a MODE method. While there appears to be no noticeable differences between the detection accuracy of the two decision fusion methods, the sensitivity and specificity of the MODE method tended to be lower and higher respectively than the OR method. That is, the OR method tended to overestimate while the MODE method tended to underestimate the number of periods of activity. These tendencies suggest that the decisions of individual sensors affect the overall activity recognition differently depending on their position (foot, shin or thigh). As such, identified periods of activity for individual sensors may need to be weighted according to their position when creating the overall activity vector for a leg.

Additionally, detector accuracy tended to be lower for infants who had more disagreement between the three codifiers examining their video data. Disagreement among these codifiers usually occurred for smaller motions made by the infant (e.g., moving only the foot or rocking the leg back and forth). These disagreements stem from differences in opinion between codifiers as to what motions qualify as a kick. To design a more accurate activity detector, it is important to understand what factors are considered when determining whether a motion is a kick.

Detector recognition was similarly affected by outside influences. Instances in the video data when the infant was moved by another person were not determined as kicking by all codifiers, but were recognized as activity by the detector.

Finally, the detector was more accurate for infants with longer session lengths. Infants with longer session lengths provided more samples for the thresholding method and shifted the calculated threshold closer to the individual's actual threshold. Thus, for long periods of observation, the accuracy of the detector is expected to increase.

### Example 2

In Example 2, machine learning methods for classifying gross kicking activity for term and preterm infants is described. Different combinations of sensors are examined to determine the relative importance of each sensor to gross activity detection. Additionally, methods to correlate infant age to the amount of time an infant performs unilateral versus bilateral kicking and time an infant is at rest are described. For preterm infants, this same relationship is examined using birth age and adjusted age. From this comparison, which age is a better predictor for movement breakdown may be determined. For gross activity recognition, it was determined that a sensor placed on the thigh was less important to overall recognition than a sensor placed on the foot or shin. Additionally, a sensor placed on the foot tended to be the most accurate on its own while the thigh sensor tended to be the least accurate. For the relationship between infant age and movement breakdown, it was determined that the amount of time spent at rest increases as age increases. Furthermore, the amount of time spent performing bilateral kicking decreases at a more rapid rate than unilateral kicking as age increases. Finally, how this relationship changes over time for infants observed over multiple months is described.

Spontaneous kicking is one of the earliest displays of motor skills and is an important precursor to later voluntary motor control [7]. Abnormal neuromotor function later in life is indicated by abnormal neuromotor function displayed through spontaneous kicking [8-9]. The direct observation of an infant's spontaneous kicking early in life can be used to detect the development of neurodevelopmental disorders like cerebral palsy (CP). However, abnormalities in spontaneous movements are not well defined and are not readily observable through traditional functional motor milestone assessments.

Example 2 describes a system and methods for the early detection of delays in infant motor development and developing CP through the extended observation of infant spontaneous kicking outside of the clinical setting. Infant kicking kinematic data is gathered over long periods of time using sensors attached to the limb segments of the infant's legs (see e.g., FIG. 5). This data is used to compute kicking kinematics and determine various features of infant spontaneous kicking. These features are used to determine a kinematic suite that describes the characteristics of typical spontaneous kicking at different months of development. With this suite, an infant is considered motor developmentally delayed if their spontaneous kicking displays characteristics typical of a younger infant.

Example 2 focuses on detecting periods of rest, unilateral activity, and bilateral activity. The ability of different machine learning algorithms to identify periods of kicking activity when different combinations of sensors are present is examined. From this, the most accurate classifier for this application, as well as the ability of different combinations of sensors to determine kicking activity, are determined. Additionally, infant age is related to the movement breakdown. A linear regression is fit to this relationship to develop a discriminative model to predict infant developmental age from movement breakdown.

Related Work

Automated Analysis of Infant Motor Development

Numerous techniques have been developed to observe and analyze various aspects of infant motor development. Multiple approaches involve the use of specialized equipment like depth cameras and motion tracking systems to gather infant movement data. Depth cameras have been used to gather infant movement data for analyzing kinematic motion and for infant pose estimation [10-12]. Olsen et al. used motion capture to gather infant pose data and track infant movement [13]. Karch et al. used electromagnetic tracking to examine upper and lower limb motions of infants [14]. Electromagnetic tracking was also used in [15] to track fidgety movements in 3D space. The methods that utilize this specialized equipment provide precise spatial tracking of infant spontaneous movement. However, such approaches and others like them require a controlled environment and expensive equipment, making these approaches unsuitable for use outside of a laboratory or clinical setting.

Other approaches utilize optical devices like video cameras to analyze infant motion data. Oftentimes, markers are placed on the infant to aid in tracking the infant's movements. In [16], baseline data of early spontaneous movement in preterm infants were established from the kinematic analysis of video data with joint markers on the infants' lower limbs. Other approaches attempt to track infant motion and identify at risk infants using marker-less video data [17]. Adde et al. utilized motiongrams to capture general movement patterns without the need of markers [18]. Stahl et al. used motion trajectories from markerless video data to analyze spontaneous movements using optical flow and wavelet analysis [19]. Das et al. tracked infant kicking from video data to collect kinematic data to identify periods of simultaneous and non-simultaneous movements [20]. However, these methods often make assumptions about the position of the joints and require a specific configuration between the camera and the infant being filmed. Additionally, these methods are oftentimes not robust to occlusions making these approaches non-ideal for usage outside of a clinical or laboratory setting.

Other approaches use wearable technology or clothing embedded with sensors to gather infant movement data. Smith et al. determined the daily quantity of infant leg movement from infant kicking data gathered over the course of a full day to determine the daily kicking sequence [21]. Accelerometer data was used in [22] to identify motor milestones in infants and identify at risk infants. Due to their low cost and ability to be utilized in multiple settings, these approaches are suitable for in-home usage. However, these approaches are limited as they require a strict definition of which movements qualify as a kick.

To enable the early detection of delays in infant motor development and developing CP, the system and methods of Example 2 allow for a longer observation time than typically allowed in a clinical setting than those proposed in the related work above. The advantages of wearable technology to enable observation in the home and thus maximize observation time are used. Additionally, the shortcomings oftentimes associated with wearable technology in this space are addressed by the systems and methods of Example 2. Rather than restricting analysis to movements that follow a strict definition, the methodology of Example 2 allows for the analysis of a multitude of spontaneous movements. These movements can aid in the determination of developmental age and would otherwise be discarded.

Activity Detection from Multiple Sensors

There are multiple approaches available to combine information gathered from multiple sensors and derive an overall decision. The first subset of these approaches is called sensor fusion, in which features from the data of multiple sensors are combined into a feature vector that is used to reach an overall decision [23]. The second subset of these approaches is called decision fusion, in which the decisions of multiple sensors are combined to reach an overall decision. Traditional sensor fusion methods assume that the feature vectors are complete; that is, it is assumed that data is not missing. Though there exist methods to estimate missing data to form complete feature vectors, these methods can lead to large errors in overall classification [24]. In comparison, data fusion methods tend to be more robust to missing data. To account for a missing sensor decision, the rule to reach an overall decision can be easily adjusted. However, the optimal rule to combine individual sensor decisions is not always known.

Example 1 used a Stance Hypothesis Optimal Detector (SHOD) with an automated thresholding method to determine instances of activity for term and low-risk preterm infants. Identified periods of activity for the individual sensors were then combined to determine overall activity for the leg [23]. In Example 1, two methods were considered to determine the overall activity for the leg. The first method, OR, dictated an instance of activity if a single sensor detected activity. This method tended to overestimate the instances of activity and the activity detector had a relatively large number of false positives which negatively impacted the overall classification accuracy. To address this issue, the second method, MODE, dictated an instance of activity if two or more sensors detected activity. The number of false positives decreased using the second method, however there was no significant difference between the accuracy of the two methods. From Example 1, it was determined that a better understanding of the importance of individual sensors to overall activity detection may be beneficial. With this information, a smarter decision fusion/sensor fusion approach can be determined to improve the accuracy of the detector.

To push forward the state-of-the-art in this domain, Example 2 describes multiple machine learning assessment methods for identifying periods of kicking activity extracted from infant kicking data. Specifically, the relative importance of each sensor to gross activity detection for data acquired from term and low-risk preterm infants can be determined. Additionally, methods that correlate infant age to the amount of time an infant performs unilateral versus bilateral kicking are described. For preterm infants, this same relationship is examined using birth age and adjusted age. Finally, how this relationship changes over time for infants observed over multiple months is examined.

Methods

To enable the early detection of neurodevelopmental motor disorders in children, the system can: allow for a longer observation time than typically allowed in a clinical setting; be adaptable to variations in infant size and age; and provide an objective, quantifiable metric of infant motor development.

TABLE V
Infant Demographics

| | | Gestation | Number of |
|---|---|---|---|
| Infant | Sex | Age (weeks) | Sessions |
| B | F | 38 | 2 |
| C₁ | F | 32 | 1 |
| C₂ | M | 32 | 1 |
| D | F | 38 | 3 |
| E | F | 39 | 2 |
| F | F | 34 | 4 |

In Table V, gestation age is the number of weeks the infant was in the womb prior to birth. Infants with a gestation age less than 37 weeks are considered preterm. Number of sessions indicates the number of times an infant was sampled. Subscripts on infant identifier denote multiple births.

Example 2 uses an infant sensor suit (see e.g., FIG. 5) to gather motion data associated with an infant's spontaneous kicking patterns. Collected data is then analyzed to determine instances of kicking activity as the first measure for calculating infant kicking kinematic data over long periods of time.

TABLE VI
Infant Session Information

| | | | Age at | Adjusted |
|---|---|---|---|---|
| | Session | Session | Testing | Age |
| Infant | Date | Length | (Months) | (Months) |
| B | Sep. 14, 2017 | 14 m 56 s | 3 | — |
| | Oct. 26, 2017 | 23 m 30 s | 4.5 | — |
| C₁ | Oct. 14, 2017 | 14 m 39 s | 5 | 0.5 |
| C₂ | Oct. 14, 2017 | 5 m 11 s | 5 | 0.5 |
| D | Nov. 3, 2017 | 10 m 59 s | 2 | — |
| | Feb. 8, 2018 | 17 m 59 s | 5 | — |
| | May 15, 2018 | 13 m 35 s | 8 | — |
| E | Dec. 12, 2017 | 15 m 25 s | 2 | — |
| | Jan. 30, 2018 | 9 m 18 s | 3.5 | — |
| F | Dec. 24, 2017 | 5 m 8 s | 2 | 0.5 |
| | Apr. 8, 2018 | 16 m 45 s | 5 | 3.5 |
| | Jun. 3, 2018 | 13 m 36 s | 7 | 5.5 |
| | Jul. 1, 2018 | 8 m 30 s | 8 | 6.5 |

In Table VI, ages are rounded to the nearest half month. Adjusted age is only calculated for infants born preterm. Subscripts on infant identifier denote multiple births.

Infant Sensor Suit and Data Collection

The system couples a Bluetooth-connected infant sensor suit with a data collection app, resident on a mobile device to enable ease-of collection in the home. The infant sensor suit pairs infant pants and rattle socks with six 6-axis IMU sensors powered by a coin cell battery (MbientLab's MetawearC). The suit incorporates 3 sensors per leg, placed on the thigh, shin, and foot to gather 3-axis acceleration and gyroscope data for each of the limb segments (FIG. 5) [36]. These sensors utilize Bluetooth Low Energy (BLE) technology for data transfer to our custom app. The app allows clinicians or parents to gather data from the sensor suit while monitoring battery life and connectivity of each sensor.

Data Collection Procedure

8 months (0.5 and 8 months adjusted age) were observed for this study (Tables V and VI). Three of the eight infants were born full term while the remaining three infants were born premature but considered low risk. Low risk, preterm infants were defined as infants born at a gestational age between 32 to 37 weeks with no severe respiratory distress during birth and no existing Grade III or IV intraventricular hemorrhage after birth [24]. For observation in the home, infants were placed supine on a flat, padded surface while wearing the infant sensor suit. The infant's legs were momentarily held stationary at the beginning of each kicking session. For this baseline study, this step allowed later calibration of a zero-point time stamp with respect to quantifying the performance of our algorithms.

The infant was then encouraged to kick by providing stimulation consisting of verbal gestural cues and presentation of physical play objects. Stimulation was provided until it was determined that the infant needed to rest. That is, the kicking session continued until the parent or clinician requested a rest period or if the infant showed any form of agitation or distress. During kicking, acceleration and angular rate data were collected at a sampling rate of 100 Hz from the embedded infant suit sensors. Several periods of data were collected with each session yielding up to 20 minutes of kicking data. Session length varied between infants due to infant emotional state; the average session length was 14 minutes. Each session was also filmed using a video timestamping application for the creation of truth data.

Construction of Ground Truth

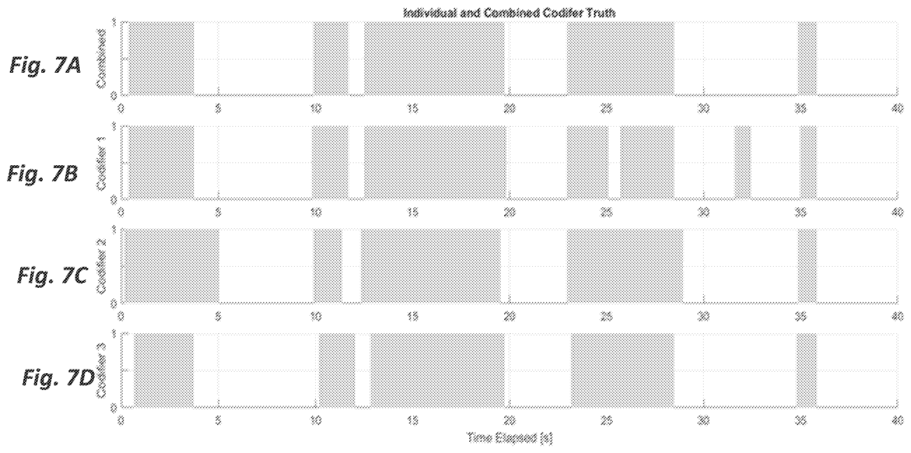

Three independent coders were separately tasked to codify instances of leg motion activity in the timestamped videos. Each coder was asked to identify moments of time when the infant's leg was moving. Results from the coders were resampled to match the sampling rate of the data collection app and then used to construct a truth vector quantifying motion activity for each kicking session. If there was disagreement in motion activity between the coders, the most popular identifier (2 out of 3) at that timestamp was used to construct the truth vector (FIGS. 7A-7D).

Coders were not given any specific instructions regarding which limb segment motion to prioritize. They were also not given instructions regarding interferences to or physical influencers of the infant's movements. These instances include occurrences where another individual, such as the parent, physically moved the infant or the infant's legs during an observation session.

Construction of Feature Vector for Activity Detection

To account for local dependencies between data points, a sliding window with a width of 250 ms and step of 100 ms was used for each segment of data. These values were determined through previous experiments to optimize the accuracy of kicking detection while compensating for sensor noise. A SHOD was used to create the feature vectors for the activity detector. SHOD is a magnitude-based method that uses acceleration and angular rate data to increase the precision and accuracy of an activity detector [17]. The SHOD value for each window was calculated and associated with the timestamp value at the center of the window (also called an instance of data). Then, these values were combined to create the figure of merit vⱼ for each sensor per leg. An explanation of the computation of vⱼ and definition of constants is given below with regard to Eq. (7). The figures of merit for each sensor or combinations therein were used to create a feature vector V for the activity detector. A feature vector was constructed for each of the infant's legs for each window of data.

Gross Activity Detection

The relative importance of each sensor to the overall detection results can be determined. Cases where all 3 sensors per leg are present, where data from two sensors are present, and where data from one sensor is present are compared. The ability of three different machine learning algorithms to identify periods of kicking activity with different combinations of sensors is examined to ensure that any trends observed are not dependent upon the classifier used.

Approaches for Activity Detection

In Example 2, three approaches were used to detect periods of activity: a thresholding method, a K-nearest neighbors (KNN) supervised learning method, and a Gaussian mixture model (GMM) unsupervised learning method. For the thresholding method and the KNN supervised learning method, a leave-one-out approach was used for training. In the leave-one-out approach, a segment of data is left out during training and the remaining segments of data are used to train the classification model. Detection results are then reported on the left-out segment of the data to evaluate the ability of the model to generalize to unseen data. For the GMM unsupervised learning method, a model was created for each segment of data without providing truth labels with respect to the detection output for that segment. Specific details for each method are detailed as follows.

Thresholding:

A thresholding method assumes that a data set can be optimally separated into two distinct classes or groups. In this method, a threshold γ from a set of training data that separates the data into two distinct classes is determined. As γ cannot perfectly separate the two classes, γ is chosen to maximize a desired classification metric (e.g. accuracy, specificity, etc.) when data in each separated class is compared to the baseline truth data [26]. In the leave-one-out approach, γ for each segment of data is determined. Then the averaged γ across the segments of data is used as the threshold for the left-out segment.

γ was specified as the point of maximum efficiency for each of the remaining segments of data as computed from the receiver operating characteristic (ROC) curve. Efficiency is a weighted average of sensitivity and specificity. Thus, the point of maximum efficiency represents the cutoff that maximizes both sensitivity and specificity. Accuracy was not used as the specified metric due to the tendency of the threshold being biased based on the larger frequency of samples from one group over another. For example, the threshold would be biased when using accuracy as the optimized metric if the infant was not moving for a significant portion of the data segment

In activity detection, the two groups for classification are as follows: a negative instance of activity (or no motion) and a positive instance of activity (or movement). For a given instance of testing data xᵢ, a positive instance of activity was indicated if

∥V(xᵢ)∥>γ(1),

where V(xᵢ) is the feature vector associated with instance xᵢ. If the above condition was not satisfied, a negative instance of activity was indicated for xᵢ. ∥V(xᵢ)∥ was calculated by taking the Euclidean norm.

K-Nearest Neighbors (KNN):

The KNN method is an unsupervised, non-parametric method for classification. KNN uses the training dataset directly to make predictions for new unseen data. For a new instance xᵢ, a prediction is made by searching through the entire training set for the K most similar instances or neighbors [27]. A distance metric is used to determine which instances in the training dataset, y, are most similar to the new instance xᵢ. For this work, the distance metric, d(xᵢ, yᵣ), is defined using a Euclidean distance:

d(xᵢ,yᵣ)={square root over (Σⱼ₌₁ⁿ(vⱼ(xᵢ)−vⱼ(yᵣ))²)} (2),

where n is the number of dimensions of the feature vector (the number of sensors present) and yᵣ represents a single instance in the training dataset to which the testing instance xᵢ is being compared. The K nearest neighbors for xᵢ are the K instances from the training set with the smallest d (xᵢ, yᵣ).

The predicted class for xᵢ is determined as the class from the training set that had the highest frequency from the K most similar instances. That is, the class from the training set with the majority of the K nearest neighbors is taken as the prediction for the new instance. The classifier in this work uses the K=50 nearest neighbors from the training set to determine the instance of activity for a given instance.

Gaussian Mixture Model (GMM):

A GMM is a probabilistic model used to represent the presence of subpopulations, or groups, within a larger population. This method constitutes a form of unsupervised learning and thus does not require a set of labeled observed (training) data to identify these groups (also known as components of the GMM) [28, 29]. GMM assumes that the individual feature vectors V(xᵢ) ∈V are derived from a mixture or sum of a finite number of Gaussian or normal distributions with unknown parameters. These individual distributions model the distribution of the data, the probability density functions (pdf's) within the different groups. The overall mixture model p(V) has the form:

$$\begin{matrix}p ( V ) = {\sum }_{k = 1}^{K} {ϕ}_{k} N ( V \vert {μ}_{k} , {Σ}_{k} ) & ( 3 ) \\ {\sum }_{k = 1}^{K} {ϕ}_{k} = 1 , & ( 4 )\end{matrix}$$

where N (V|μₖ, Σₖ) is the pdf of group k, μₖ And Σₖ Are the mean and covariance matrix specifying the normal distribution, and K is the number of groups. The group mixture weights, ϕₖ for group k, are constrained to sum to 1 so that the total pdf normalizes to 1. Finally, the dimension of μₖ and Σₖ are determined by the dimension of the feature vectors V(xᵢ):

An expectation-maximization algorithm is used to iteratively estimate the model parameters (μₖ, Σₖ, ϕₖ) for each normal distribution in the mixture model. The expectation step determines the expected group assignment Cₖ for each instance xᵢ is calculated given the model parameters (i.e. p(Cₖ|V(xᵢ),\hat{μ},\hat{Σ}, \hat{ϕ})). The maximization step then maximizes the expectations calculated in the expectation step and updates the model parameters. This process repeats until the algorithm converges resulting in a maximum likelihood estimate.

With the estimated model parameters, data can be clustered by assigning each datum to its most likely cluster assignment That is, cluster assignment is by the most likely group assignment. The probability that an instance xᵢ belongs to a certain component assignment Cₖ is calculated by:

$$p ( {C}_{k} \vert V ( {x}_{i} ) ) = \frac{{ϕ}_{k} N ( V ( {x}_{i} ) \vert )}{{\sum }_{r = 1}^{K} {ϕ}_{r} N ( V ( {x}_{i} ) \vert )} , \qquad ( 5 )$$

where the model parameters specifying the normal distributions are the estimated parameters from the expectation-maximization algorithm.

A GMM is estimated such that the model clusters the data into two groups over N replicates or repetitions. Then, the estimated model most likely to describe the data is selected from the N replicates and used to assign instances of activity to each timestamp. N=15 was chosen to ensure that at least one replicate converged while not being too computationally intensive.

Activity Detection with Combinations of Sensors

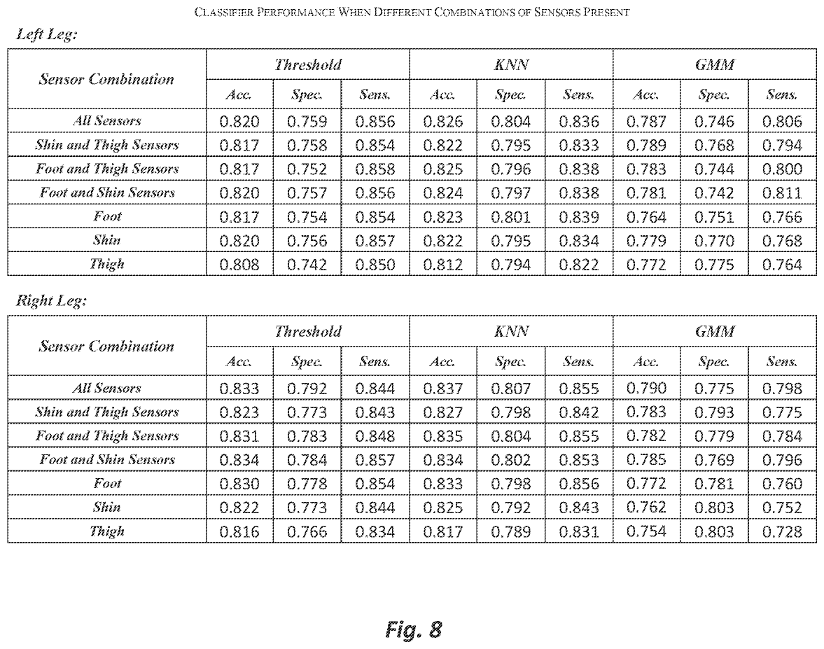

Example 2 determines the robustness of the gross activity detection approaches relative to the presence or loss of sensor data acquired from term and low-risk preterm infants. As such, different combinations of sensor loss are considered when evaluating the three methods for activity detection to ensure that any trend observed is not dependent upon the classifier used. The results are included in the table shown in FIG. 8.

All Sensors Present:

The performance of the different activity detection approaches discussed above (e.g., thresholding, KNN, and GMM methods) when all three sensors per leg are present are examined. This serves as a baseline on the optimal performance associated with the three different approaches. In the baseline assessment, the individual sensor figures of merit are concatenated to create a three-dimensional feature vector at each instance (e.g. V(xᵢ)=[v_{thigh}(xᵢ), vₛₕᵢₙ(xᵢ), v_{foot}(xᵢ)]).

Two Sensors Present:

The impact a missing sensor has on overall activity recognition is examined. That is, if data from one sensor is lost, how well does the algorithm perform with the remaining two sensors. The case where the shin and thigh sensor are present, the case where the shin and foot sensor are present, and the case where the thigh and foot sensor are present are examined. For the sensors present, the individual sensor figures of merit are concatenated to create a two-dimensional feature vector at each timestamp (e.g. V(xᵢ)=[v_{thigh}(xᵢ), vₛₕᵢₙ(xᵢ)]).

One Sensor Present:

Algorithm robustness is examined by measuring its performance when only one sensor is present. This provides an indication of the reliability of the various approaches and their dependency on individual sensor placement. It also serves as an indication of the relative importance of each sensor to overall detection of motor activity.

TABLE VII
Comparison of Linear Models for Motion States

| Motion State | | R² | SSE |
|---|---|---|---|
| No | Birth Age | 0.66 | 1,071.93 |
| Motion | Adj. Age | 0.75 | 791.60 |
| Unilateral | Birth Age | 0.02 | 703.26 |
| | Adj. Age | 0.07 | 651.445 |
| Bilateral | Birth Age | 0.67 | 904 |
| | Adj. Age | 0.62 | 1,047.04 |

In Table VII, linear models displayed on their corresponding plots in FIGS. 9A-9C and 10A-10C. The predicted value for the linear model corresponds to the percent of time spent in a motion state while the predictor (age) corresponds to either the infant's birth age or adjusted age.

Results for Gross Activity Detection

The table is FIG. 8 depicts how well each algorithm predicts overall leg movement using the different combinations of available sensors. Values displayed are the average performance of the classifier over all infants over all testing sessions. Though no statistically significant claims can be made due to the small sample size, there were numerous observed trends.

Overall, the highest accuracy was achieved when all three sensors were present. For the threshold method, a comparable detection accuracy was reached when the foot and shin sensors were present. In general, the omission of the foot or shin sensor impacted accuracy more than the omission of the thigh sensor. Additionally, the foot sensor tended to be the most accurate sensor for detecting activity on its own though in some instances, the shin sensor was more accurate. In general, the thigh sensor was the least accurate for detecting accuracy on its own. In terms of gross activity recognition, the thigh sensor was found to be less important to overall recognition than the foot or shin sensor.

All methods and combinations of sensors have higher sensitivity than specificity. As such, all combinations of sensors were able to identify positive instances of activity better than negative instances of activity. Finally, of the three detection methods, the KNN method performed the best for all sensor combinations.

Relationship Between Movement Breakdown and Age

The method for determining correlations between characterization of the kicking activity and the infant's age is described below. The objective for developing such a model is important in identifying features of normative kicking, leading to early identifications of delayed or atypical kicking profiles. For characterizing kicking activity, movements can be decomposed into four states (sometimes referred to herein as “motion states”) and the proportion of time an infant spends in each of the states can be determined. The four states are: at rest (no motion), unilateral motion (dominant leg movement only), and bilateral (both legs moving). In the unilateral motion state, the dominant leg depends on the individual. That is, percentages reported are either left leg unilateral motion or right leg unilateral motion depending on which leg is dominant for the infant (e.g., which state between unilateral left and unilateral right the infant spends more time in). Additionally, the development of preterm infants is generally measured on an adjusted scale. That is, preterm infants of a certain adjusted age are typically compared to term infants of that birth age. However, it is unclear whether a preterm infant should be considered by their adjusted age or their birth age in this relationship. In Example 2, preterm infants are elevated based on their adjusted age and their birth age.

Descriminative Model Fitting

To develop a model that defines the relationship between characterization of kicking activity and infant age, the percent of time the infant spent in each motion state was plotted against the infant's age. A linear regression was fit to each motion state to predict percentage of time given an infant's age. A sum of squares error (SSE) was then calculated for each motion state to indicate how well the linear model represented the data:

SSE=Σᵢ₌₁ⁿ(yᵢ−ŷᵢ)² (6),

where yᵢ is an observed percentage and ŷᵢ is the predicted percentage from the linear model. When the SSE's from two models are compared, the smaller SSE indicates the model with higher predictive power. The coefficient of determination, R², is also reported for each model. R² indicates the percentage of variability in the response variable that is explained by the linear model. Generally, a higher R² indicates a better fitting model though this is not guaranteed. As such, it is important to consider R² in addition to another metric for goodness of fit like the SSE.

Results for Correlating Movement Breakdown and Infant Age

FIGS. 9A-9C and 10A-10C plot the observed percentage breakdowns versus infant adjusted age and infant birth age, respectively. The breakdown of kicking activity reported is from activity as detected from the KNN classification method when all three sensors were present. Orange×markers represent observations from term infants while blue+markers represent observations from preterm infants. Additionally, the linear models are displayed. In these results, it is observed that the no motion class had a positive relationship with infant age while the unilateral motion class and the bilateral motion class had a negative relationship with age. Furthermore, the slope of the regression for the bilateral motion class is steeper than that of the unilateral motion class. Thus, as the infant ages, the amount of gross motor activity of the lower limbs decreases overall while instances of bilateral motion decrease more rapidly than instances of unilateral motion.

Table VII reports the SSE's of the six linear models. Within each motion state, the SSE of the linear model associated with birth age is compared to that of the SSE of the linear model associated with adjusted age. Generally, the SSE for the models using the adjusted age of the premature infants was lower except for the bilateral motion state. Additionally, the R² value was higher for the adjusted age in the no motion state and the unilateral motion state while the R² value for the bilateral motion state was higher for birth age. From the data of Example 2, evaluating premature infants at their adjusted age generally results in smaller residuals and higher coefficients of determination and thus a more accurate prediction.

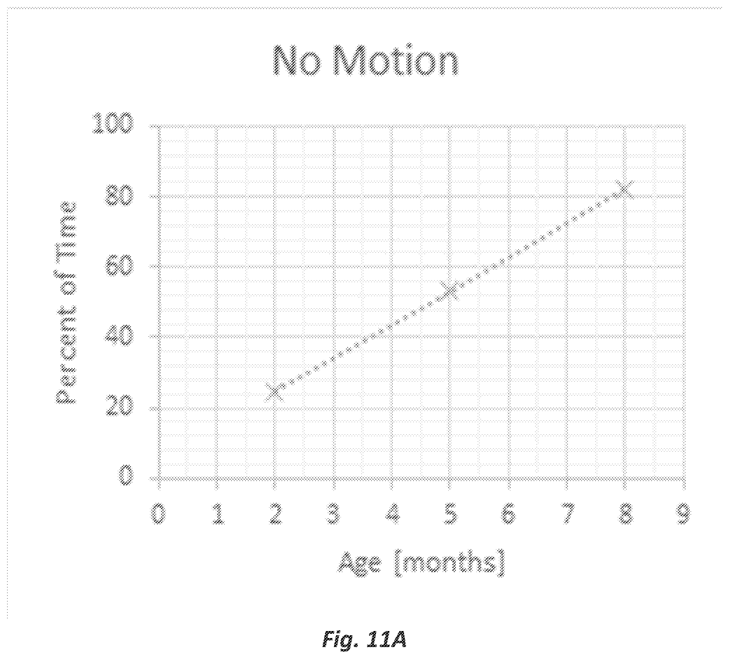

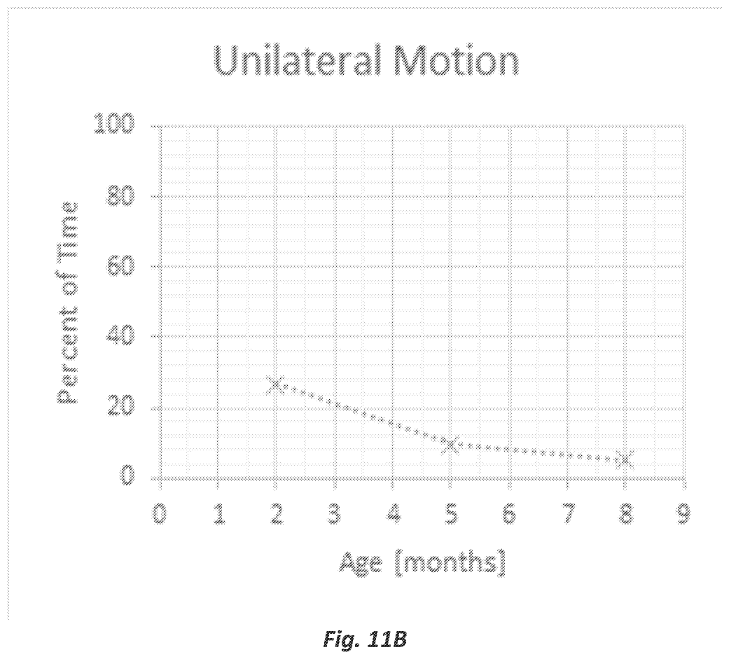

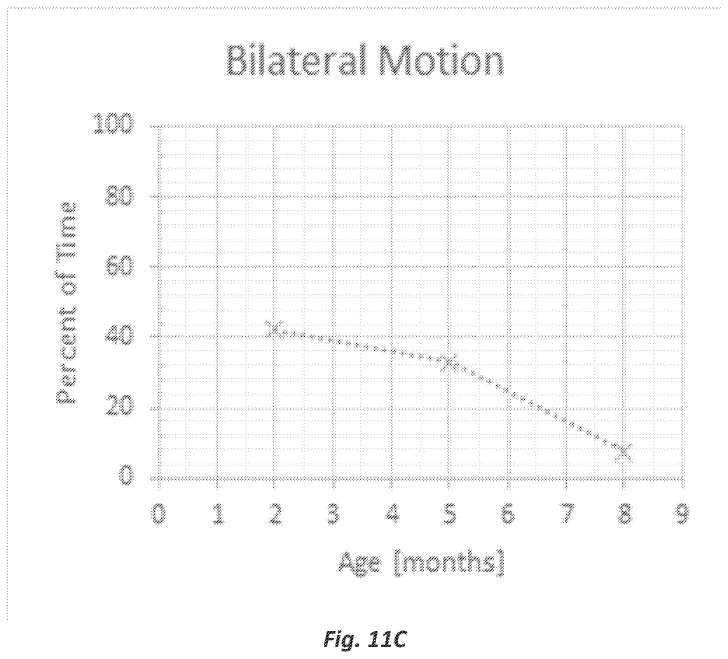

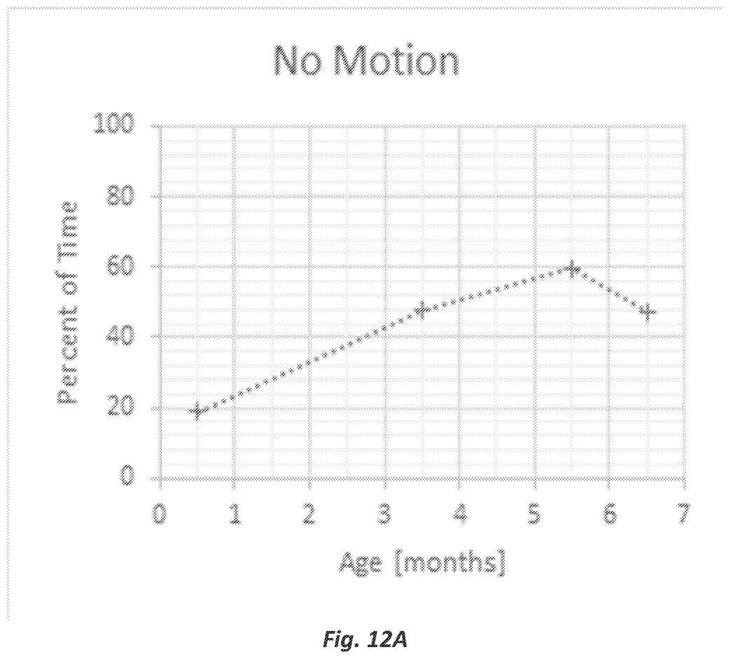

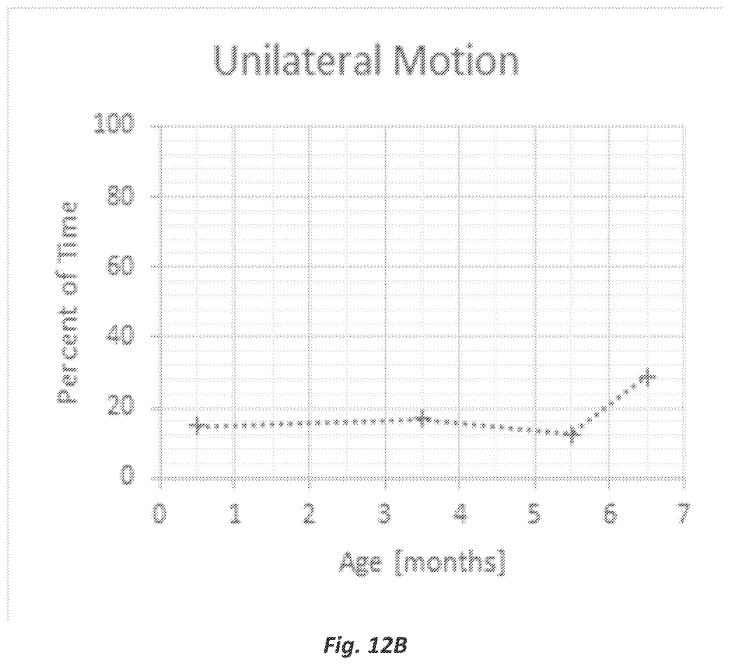

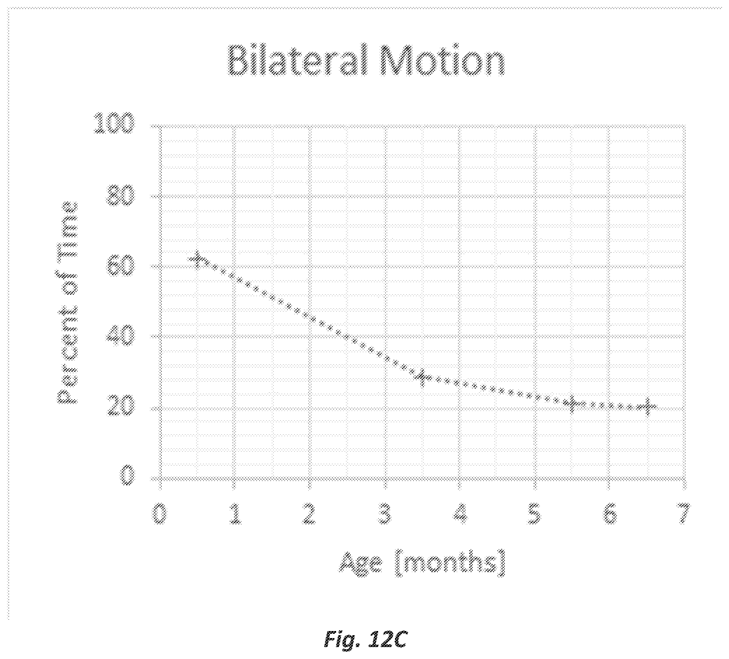

FIGS. 11A-11C plot the observed percentage breakdowns versus infant age for infant D and FIGS. 12A-12C plot the observed percentage breakdowns versus infant adjusted age for infant F. Infant D was born to term while infant F was born 6 weeks preterm. For infant D, the amount of time the infant spends at rest increases with age while the amount of time the infant spends performing bilateral or unilateral movement decreases with age. Similar trends are observed for infant F. However, there is a distinct increase in percent time for unilateral motion and a distinct decrease in percent time for no motion from 5.5 month to 6.5 months adjusted age that goes against the trend for the overall group.

SHOD Method

SHOD is a magnitude-based method that uses both acceleration and angular rate data to increase the precision and accuracy of an activity detector.

$$v ( {x}_{i} ) = \frac{1}{N} {\sum }_{k = 1}^{N} ( \frac{1}{{σ}_{a}^{2}} {‖ {a}_{k} - g \frac{{\overline{a}}_{n}}{‖ {\overline{a}}_{n} ‖} ‖}^{2} + \frac{1}{{σ}_{ω}^{2}} {‖ {ω}_{k} ‖}^{2} ) \qquad ( 7 )$$

where: n refers to a specific frame, centered at instance xᵢ, which represents the content (acceleration and angular rate data) of a sliding window; aₖ and ωₖ are the acceleration and angular rate vector respectively for observation k of a specific frame n; āₙ is the mean of the acceleration vector of a specific frame n; g is the magnitude of acceleration due to gravity (1 g).

The constants determined are as follows: N=25 is the number of samples in a frame determined by the desired length of the frame in seconds multiplied by the sampling frequency; σₐ²=3.24×10⁻⁶, variance of the acceleration signal noise, and σ_{ω}²=0.0049, variance of the angular rate signal noise, are determined from specifications of the IMU.

### Conclusion

In Example 2, machine learning methods for classifying gross kicking activity for term and preterm infants are described and different combinations of sensors are examined to determine the relative importance of each sensor to gross activity detection. While the highest overall accuracy was achieved when the foot, shin, and thigh sensor were all present, results indicate that for gross activity detection, the omission of a sensor placed on the thigh did not impact overall recognition as much as the omission of a sensor placed on the foot or shin. Individually, a sensor placed on the foot tended to be the most accurate on its own while the thigh sensor tended to be the least accurate.

Methods to correlate infant age to the amount of time an infant is at rest, performing unilateral activity, or performing bilateral activity is also described. It was determined that as the amount of time they spent at rest increases as age increases. Furthermore, the amount of time spent performing bilateral kicking decreases at a more rapid rate than unilateral kicking as age increases.

Finally, how this relationship changes over time for infants observed over multiple months is examined. The trends observed for the three motion states when examining the group are reflected in the analysis of the two infants (one term and one premature) over multiple months.

To enable the early detection of delays in infant motor development and developing CP through extended observation of infant spontaneous kicking outside of the clinical setting. Characteristics of gross movement activity such as movement breakdown and the kinematic characteristics therein could serve as one feature to detect motor development delays.

### REFERENCES

### Example 1

[1] A. Herskind, G. Greisen, and J. B. Nielsen. “Early identification and intervention in cerebral palsy,” Developmental Medicine & Child Neurology, v. 57 no.1, pp. 29-36, 2015.

[2] D. Bryant et al., “An infant smart-mobile system to encourage kicking movements in infants at-risk of cerebral palsy,” in IEEE Workshop on Advanced Robotics and its Social Impacts (ARSO), Austin, Tex., 2017.

[3] E. Rogers et al., “Smart and Connected Actuated Mobile and Sensing Suit to Encourage Motion in Developmentally Delayed Infants,” in ASME Design of Medical Devices Conf., Minneapolis, Minn., 2015.

[4] K. Subramanyam et al., “Soft Wearable Orthotic Device for Assisting Kicking Motion in Developmentally Delayed Infants,” J. of Medical Devices, v. 9 no. 3, 2015.

[5] M. Hadders-Algra, “Early diagnosis and early intervention in cerebral palsy,” Frontiers in Neurology, v. 5, 2014.

[6] J. P. Piek and N. Gasson, “Spontaneous kicking in fullterm and preterm infants: Are there leg asymmetries?,” Human Movement Science, v. 18 no. 2, pp. 377-395, 1999.

[7] F. B. Palmer, “Strategies for the early diagnosis of cerebral palsy.” The J. of pediatrics, v. 145 no. 2, 2004.

[8] M. Bax et al., “Proposed definition and classification of cerebral palsy,” Developmental Medicine and Child Neurology, 2005.

[9] M. D. Olsen et al., “Using Motion Tracking to Detect Spontaneous Movements in Infants,” in Scandinavian Conference on Image Analysis, pp. 410-417. Springer, Cham, 2015.

[10] D. Karch et al., “Kinematic assessment of stereotypy in spontaneous movements in infants,” Gait & Posture, v. 36 no. 2, 2012.

[11] C. B. Heriza, “Organization of leg movements in preterm infants,” Physical Therapy, v. 68 no. 9, pp. 1340-1346, 1988.

[12] H. Rahmati et al., “Video-based early cerebral palsy prediction using motion segmentation,” Ann. Int. Conf. of the IEEE EMBC, 2014.

[13] N. Hesse et al., “Body pose estimation in depth images for infant motion analysis,” Ann. Int. Conf. of the IEEE EMBC, 2017.

[14] M. Serrano et al., “Lower Limb Pose Estimation for Monitoring the Kicking Patterns of Infants,” Ann. Int. Conf. of the IEEE EMBC, 2016.

[15] D. Gravem et al., “Assessment of Infant Movement With a Compact Wireless Accelerometer System,” ASME J. of Medical Devices, 2012.

[16] B. A. Smith et al., “Daily Quantity of Infant Leg Movement: Wearable Sensor Algorithm and Relationship to Walking Onset,” Sensors, v. 15 no. 8, pp. 19006-19020, 2015.

[17] A. Olivares et al., “Detection of (In)activity Periods in Human Body Motion Using Inertial Sensors: A Comparative Study,” Sensors, 2012.

### Example 2

[1] A. Herskind, G. Greisen, and J. B. Nielsen. “Early identification and intervention in cerebral palsy,” Developmental Medicine & Child Neurology, v. 57 no.1, pp. 29-36, 2015.

[2] E. Rogers et al., “Smart and Connected Actuated Mobile and Sensing Suit to Encourage Motion in Developmentally Delayed Infants,” in ASME Design of Medical Devices Conf., Minneapolis, Minn., 2015.

[3] K. Subramanyam et al., “Soft Wearable Orthotic Device for Assisting Kicking Motion in Developmentally Delayed Infants,” J. of Medical Devices, v. 9 no. 3, 2015.

[4] D. Bryant et al., “An infant smart-mobile system to encourage kicking movements in infants at-risk of cerebral palsy,” in IEEE Workshop on Advanced Robotics and its Social Impacts (ARSO), Austin, Tex., 2017.

[5] M. Hadders-Algra, “Early diagnosis and early intervention in cerebral palsy,” Frontiers in Neurology, v. 5, 2014.

[6] B. A. Smith et al., “Daily Quantity of Infant Leg Movement: Wearable Sensor Algorithm and Relationship to Walking Onset,” Sensors, v. 15 no. 8, pp. 19006-19020, 2015.

[7] J. P. Piek and N. Gasson, “Spontaneous kicking in fullterm and preterm infants: Are there leg asymmetries?,” Human Movement Science, v. 18 no. 2, pp. 377-395, 1999.

[8] Jeng, L. Chen, K.Yau et al., “Kinematic Analysis of Kicking Movements in Preterm Infants With Very Low Birth Weight and Full-Term Infants,” Physical Therapy, v. 82 no.2, pp. 148-159, 2002.

[9] N. Hesse et al., “Body pose estimation in depth images for infant motion analysis,” Ann. Int. Conf. of the IEEE EMBC, 2017.

[10] M. Serrano et al., “Lower Limb Pose Estimation for Monitoring the Kicking Patterns of Infants,” Ann. Int. Conf of the IEEE EMBC, 2016.

[11] M. D. Olsen et. al.,“Using Motion Tracking to Detect Spontaneous Movements in Infants,” in Scandinavian Conference on Image Analysis, pp. 410-417. Springer, Cham, 2015.

[12] D. Karch et al., “Kinematic assessment of stereotypy in spontaneous movements in infants,” Gait & Posture, v. 36 no. 2, 2012.

[13] R. Parsa et al., “Features for Movement based Prediction of Cerebral Palsy,” in Department of Engineering Cybernetics, Norwegian University of Science and Technology, 2009.

[14] C. B. Heriza, “Organization of leg movements in preterm infants,” Physical Therapy, v. 68 no. 9, pp. 1340-1346, 1988.

[15] H. Rahmati et al., “Video-based early cerebral palsy prediction using motion segmentation,” Ann. Int. Conf of the IEEE EMBC, 2014.

[16] L. Adde et al., “Using computer-based video analysis in the study of fidgety movements,” Early Human Development, v. 85, pp.541-547, 2009

[17] A. Stahl et al., “An Optical Flow - Based Method to Predict Infantile Cerebral Palsy,” IEEE Transactions on Neural Systems and Rehabilitation Engineering, v. 20, no.4, pp. 605-612, 2012.

[18] D. Das et al., “Vision-Based Detection of Simultaneous Kicking for Identifying Movement Characteristics of Infants At-Risk for Neuro-Disorders,” IEEE ICMLA,2018.

[19] B. A. Smith et al., “Daily Quantity of Infant Leg Movement: Wearable Sensor Algorithm and Relationship to Walking Onset,” Sensors, v. 15 no. 8, pp. 19006-19020, 2015.

[20] D. Gravem et al., “Assessment of Infant Movement With a Compact Wireless Accelerometer System,” ASME J. of Medical Devices, 2012.

[21] Gao, Lei, Bourke, A. K., and John Nelson. “Evaluation of Accelerometer Based Multi-Sensor Versus Single-Sensor Activity Recognition Systems.” Medical Engineering and Physics (2014) [22] Garcia-Laencina, P. J., et al. “Pattern Classification with Missing Values using Multitask Learning,” Internation Joint Conference on Neural Networks, 2006.

[23] Fry, Katelyn E., et al. “Detection of Infant Motor Activity During Spontaneous Kicking Movement for Term and Preterm Infants Using Inertial Sensors.” IEEE Engineering in Medicine and Biology Society (EM BC), 2018.

[24] A. Olivares et al., “Detection of (In)activity Periods in Human Body Motion Using Inertial Sensors: A Comparative Study,” Sensors, 2012.

[25] T. M. Mitchell, “Machine Learning,” Mc Graw-Hill (1997).

[26] A. Moore, “Statistical Data Mining Tutorial on Gaussian Mixture Models,” www.cs.cmu.edu/^{˜}awm/tutorials, CMU (2004). B. Scherrer, “Gaussian Mixture Model Classifiers,” http://www. medialab.bme.hu/medialabAdmin/uploads, (2007).

[27] D. Gravem et al., “Assessment of Infant Movement With a Compact Wireless Accelerometer System,” ASME J. of Medical Devices, 2012.

[28] Attal, Ferhat, et al. “Physical Human Activity Recognition Using Wearable Sensors.” International Federation of Automatic Control (IFAC) (2015)

[29] B. Jeon and D. A. Landgrebe, “Decision Fusion Approach for Multitemporal Classification,” IEEE Transactions on Geoscience and Remote Sensing, vol. 37 no. 3, pp. 1227-1233, 1999.

[30] Seraji, Homayoun, and Navid Serrano, “A Multisensor Decision Fusion System for Terrain Safety Assessment.” IEEE Transactions on Robotics vol. 25 no. 1 (2009).

[31] Chen, Biao, and Pramod K. Varshney. “A Bayesian Sampling Approach to Decision Fusion Using Hierarchical Models.” IEEE Transactions on Sampling Processing (2002).

[32] Y. P. Chen and Linda Fetters. “A Comparison of the Leg Coordination Patterns of Preterm and Fullterm Infants: A Meta-Analysis.” Formosan Journal of Physical Therapy vol. 27 no. 6 (2002).

[33] R. B. Lanjewar, et al. “Implementation and comparison of speech emotion recognition system using gaussian mixture model (gmm) and k-nearest neighbor (k-nn) techniques.” Procedia Computer Science, (2015).

[34] “Detection of Infant Motor Activity During Spontaneous Kicking Movements for Term and Preterm Infants Using Inertial Sensor,” U.S. Provisional Pat. Ser. No. 62/700,781, filed Jul. 20, 2018.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

1. A computer-implemented method for detecting a neurodevelopmental disorder of an infant, comprising:
receiving motion data associated with the infant's gross motor activity;
analyzing, using a machine learning algorithm, the motion data to detect a kinematic feature;
comparing the kinematic feature to an expected relationship between the kinematic feature and infant age; and
detecting the neurodevelopmental disorder based on the comparison.

2. The computer-implemented method of claim 1, wherein the infant's gross motor activity comprises a plurality of spontaneous kicking movements.

3. The computer-implemented method of claim 1, wherein the kinematic feature is a percentage of time the infant spent in a motion state.

4. The computer-implemented method of claim 3, wherein the motion state is no motion, unilateral motion, or bilateral motion.

5. The computer-implemented method of claim 1, wherein the kinematic feature is kick frequency, spatiotemporal organization, inter-joint coordination, inter-limb coordination, phase lag, constrained movement duration, duration of movement, average acceleration, peak acceleration, joint angles, joint angle excursion, peak joint velocities, or intra-limb coordination.

6. The computer-implemented method of claim 1, wherein detecting the neurodevelopmental disorder based on the comparison comprises detecting that the infant is motor developmentally delayed for the infant's age.

7. The computer-implemented method of claim 1, wherein the machine learning algorithm is a supervised or unsupervised learning algorithm.

8. (canceled)

9. The computer-implemented method of claim 1, wherein the motion data is received from one or more sensors placed at the infant's lower limb.

10. The computer-implemented method claim 1, wherein the neurodevelopmental disorder is cerebral palsy.

11. A system for detecting a neurodevelopmental disorder of an infant, comprising:
a sensor configured for placement at the infant's lower limb; and
a computing device operably coupled to the sensor, the computing device comprising a processor and a memory operably coupled to the processor, the memory having computer-executable instructions stored thereon that, when executed by the processor, cause the computing device to:
receive, from the sensor, motion data associated with the infant's gross motor activity;
analyze, using a machine learning algorithm, the motion data to detect a kinematic feature;
compare the kinematic feature to an expected relationship between the kinematic feature and infant age; and
detect the neurodevelopmental disorder based on the comparison.

12. The system of claim 11, wherein the sensor is configured for placement at the infant's thigh, shin, or foot.

13. (canceled)

14. The system of claim 11, wherein the sensor is an inertial measurement unit (IMU).

15. The system of claim 11, further comprising a plurality of sensors configured for placement at the infant's lower limb

16. The system cvlaim 11, wherein the infant's gross motor activity comprises a plurality of spontaneous kicking movements.

17. The system of claim 11, wherein the kinematic feature is a percentage of time the infant spent in a motion state.

18. The system of claim 17, wherein the motion state is no motion, unilateral motion, or bilateral motion.

19. The system of claim 11, wherein the kinematic feature is kick frequency, spatiotemporal organization, inter-joint coordination, inter-limb coordination, phase lag, constrained movement duration, duration of movement, average acceleration, peak acceleration, joint angles, joint angle excursion, peak joint velocities, or intra-limb coordination.

20. The system of claim 11, wherein detecting the neurodevelopmental disorder based on the comparison comprises detecting that the infant is motor developmentally delayed for the infant's age.

21. The system of claim 11, wherein the machine learning algorithm is a supervised or unsupervised learning algorithm.

22. (canceled)

23. (canceled)

24. A computer-implemented method for detecting a motor developmental delay of an infant, comprising:
receiving motion data associated with the infant's gross motor activity;
analyzing, using a machine learning algorithm, the motion data to detect a kinematic feature;
comparing the kinematic feature to an expected relationship between the kinematic feature and infant age; and
detecting the motor developmental delay based on the comparison.

25-32. (canceled)
